(19) United States
(12) Patent Application Publication
(10) Pub. No.: US 2025/0107812 A1
(43) Pub. Date: Apr. 3, 2025

(54) RIGIDIZING ASPIRATION SYSTEMS AND METHODS

(71) Applicant: Neptune Medical Inc., Burlingame, CA (US)

(72) Inventors: Maya Rose EISLER, San Francisco, CA (US); Lior NOYOVITCH, Burlingame, CA (US); Garrett J. GOMES, Pleasant Hill, CA (US); Connor MULCAHY, San Francisco, CA (US); Alexander Q. TILSON, Burlingame, CA (US); Charles Steele LOVE, Santa Barbara, CA (US); Kendall CARPENTER, Redwood City, CA (US); David RAHDERT, Burlingame, CA (US)

(21) Appl. No.: 18/852,419

(22) PCT Filed: May 30, 2024
(86) PCT No.: PCT/US2024/031789

§ 371 Date: Sep. 27, 2024

Related U.S. Application Data
(60) Provisional application No. 63/505,062, filed on May 30, 2023.

(51) Int. Cl. A61B 17/22; A61B 17/00

(52) U.S. Cl. CPC A61B 17/22; A61B 17/00234; A61B 2017/00238; A61B 2017/00305; A61B 2017/00367; A61B 2017/00561; A61B 2017/22079; A61B 2217/005

(57) ABSTRACT

Methods and apparatuses, and in particular rigidizing catheters and associated components, that may be used to provide access and support to one or more cardiovascular devices, including aspiration devices. In particular, described herein are methods and apparatuses to remove clot material from a vessel, using a rigidizing aspiration sheath catheter that is adapted for use within a subject's vasculature.

### CLAIM OF PRIORITY

This patent application claims priority to U.S. provisional patent application No. 63/505,062, titled “RIGIDIZING ASPIRATION SYSTEMS AND METHODS,” filed on May 30, 2023 and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

During medical procedures, especially procedures to be performed within the vasculature, the interventional or diagnostic medical device often has to be navigated through long and tortuous anatomy to reach the site of interest. This complicated navigation and resulting pathway to the treatment site requires highly flexible catheters, which typically results in poor control of the interventional or medical device once treatment is attempted at the destination. There are different optimal stiffnesses for the different activities of navigation and treatment. Heretofore, most catheter designers have tried to solve the problem of balancing stiffness for navigation and for treatment by creating devices whose stiffness is a compromise between those different activities.

Most transcatheter procedures rely on the use of a pre-placed guidewire to direct and navigate the catheter to the desired anatomy. For example, prior to deploying a stent in the coronary artery, a guidewire is advanced through the lesion to serve as a guide for the catheter carrying the stent. However, the use of guidewires may complicate these procedures, requiring extra steps, and may not provide appropriate stability and support when performing operations within the vasculature. Guidewires may also displace the catheter being guided, particularly when navigating through tortuous anatomy. Guidewires may need to be ‘swapped’ for sequentially different stiffness guidewires, which adds time, risk and cost. Guidewire displacement, including losing guidewire position, can affect the ability to place the catheter in the correct position and can significantly extend procedural difficulty, risk, and duration and may lead to complications and iatrogenic injury.

Moreover, guidewires and guide catheters are often not effective for large bore catheters (e.g., large bore catheters suitable for treating pulmonary embolisms) because the large bore catheter may not easily track over the guidewire through tortuous anatomy (e.g., through the right atrium, the tricuspid valve, the right ventricle, the pulmonary valve, and through the pulmonary bifurcations). These larger bore catheters can be quite stiff, which, when navigated amongst tortuous anatomy, create stored energy which can be released unpredictably, causing procedural uncertainty and potentially dangerous complications including iatrogenic injury. In general, stiffer catheters require stiffer guidewires, which have more clinical risks—including perforations—as compared to more flexible guidewires.

A catheter that can be placed in the anatomy in a flexible condition may be better able to conform to the anatomy. Once rigidized, the anatomical pathway is preserved, protecting the adjacent anatomy by limiting forces that straighten the access. In one example, a pathway developed by an access catheter may begin in a state of anatomical conformation, but subsequent instruments placed within the access catheter may increase the stiffness and straighten the sheath and adjacent anatomy. Isolating and preserving the anatomical pathway reduces the potential for iatrogenic injury and hemodynamic complications associated with impingement of certain anatomical structures like heart valves.

Vascular obstructions (clots) may be removed with multiple devices, including mechanical removal, hydraulics (pressurized fluid streams) and by aspiration. Aspiration systems apply vacuum along a lumen from a proximal low-pressure source (for example, a vacuum pump) to a distal opening. Along the luminal path, systems typically incorporate features such as valve(s), gage(s), disconnect(s), flush port(s), guidewire access port(s), clot capture(s), blood capture, filter(s), and system(s) for blood re-infusion. These lumens may be constant bore, or they may have regions that expand, including distally, to capture larger clots more easily, and to increase the local vacuum suction area. The lumens may be open bore, or they may have elements therewithin that serve to process, chop, dissolve, macerate, cut, slice, or otherwise reduce the clot's local cross-sectional area, such that it is more easily transported from its distal input to its proximal output.

In some cases, other, more difficult or complicated treatments, are required. For example, pulmonary embolism treatment may require thrombolytics, which may be expensive, may have potentially deadly complications, may require an ICU stay, and are not particularly effective for larger or older clots. In some cases pulmonary embolism treatment may require surgery, which may be traumatic and expensive, including a sternotomy and bypass, and may require an extended stay in the hospital, and prolonged recovery.

There is a need for methods and apparatuses that may provide a safer and more reliable pathway to internal treatment sites, and that may allow enhanced treatments once at the site.

### SUMMARY OF THE DISCLOSURE

Described herein are methods and apparatuses, including systems and devices, and in particular catheters and associated components, that may be used to provide access and support to one or more cardiovascular procedures, including in particular, described herein are methods and apparatuses to remove material (e.g., clot material) from a vessel. For example, described herein are rigidizing aspiration sheath catheters that are adapted for use within a subject's vasculature (e.g., blood vessels, heart, etc.). As used herein vascular may include any vascular region of the body, including, but not limited, to peripheral, neurovascular, etc. These apparatuses, e.g., rigidizing aspiration sheath catheters, may include an integrated valve (e.g., hemostasis valve region) to prevent or limit blood loss during the procedure without interfering with the ability of the apparatus to rigidizing/de-rigidize. These apparatuses may be sized and shaped for insertion into the vasculature. The rigidizing aspiration sheath catheters described herein may be converted between one or more flexible states that may be readily navigated through even a tortuous anatomy, and one or more more-rigid states, in which the shape (including any bends or curves) of the rigidizing aspiration sheath catheter is locked in position. Intermediate states may also be useful at certain times during the procedure. As used herein, the flexible state may be highly flexible and the rigid state is generally more rigid than the flexible state; in some examples the rigid state may be highly rigid.

The rigidizing aspiration sheath catheters described herein may be configured to apply aspiration (e.g., suction) directly through the lumen of the rigidizing aspiration sheath catheter and/or they may be configured to receive an aspiration catheter (which, in some examples, may or may not also be rigidizing). The rigidizing aspiration sheath catheters described herein may be adapted to locking or coupling to a distal end of a suction line, using a universal coupling, e.g., mating attachment connector, that mates with a mating attachment at a distal end of vacuum line; the same mating attachment may lockingly couple with a separate aspiration catheter that may be used with, including inserted into, a rigidizing aspiration sheath catheter. In some cases an aspiration catheter (or other device) may be inserted into the lumen of the rigidizing aspiration sheath catheter while still allowing aspiration to be applied through the lumen of the rigidizing aspiration sheath catheters. For example, the apparatus may include an aspiration line that engage with the proximal end of the rigidizing aspiration sheath catheters including the hemostasis valve to allow insertion (and form a seal around the inserted) aspiration catheter or other device.

The apparatuses described herein may address the problem of balancing different stiffnesses for navigation and for treatment by configuring and using devices or systems that may dynamically modulate their stiffnesses (‘dynamically rigidizing’ or ‘dynamically stabilizing’), from high flexibility to high stiffness, throughout the procedure, potentially as a function of location along the device shaft, with a fast, facile, and indefinitely large number of transitions between these variable stiffness states. As mentioned above, these apparatuses may have significant advantages as compared with standard catheters that are not dynamically rigidizing, and typically have a set, fixed stiffness, though those fixed stiffness values can vary by zone (typically the zones change along the catheter's length). The dynamically rigidizing apparatuses (including but not limited to dynamically rigidizing catheters and other components) described herein can have regions that are selectively rigidized, or the entire shaft can be rigidized or made flexible as one cohesive entity. A dynamically rigidizing catheter can be configured to have a substantially lower proximal stiffness than a typical catheter, which can be advantageous as the proximal zone navigates through anatomy that can be tortuous, calcific, and, in some instances, can extend through atrium, ventricles, and valves. In these instances, lower baseline stiffness puts lower load on localized anatomical features.

In general, the methods and apparatuses described herein are particularly useful for vascular indications, including cardiovascular, peripheral vascular, cerebrovascular, neurovascular, pulmonary vascular, thoracic vascular, abdominal vascular, lymphatic vascular, renal vascular, and/or genitourinary vascular. The rigidizing aspiration sheath catheter may be dynamically rigidized, e.g., switched between a rigid state and a flexible state. The rigidizing aspiration sheath catheter may also include a hemostatic valve region at the proximal end. The hemostatic valve region may be integrated into the rigidizing aspiration sheath catheter. In some examples the rigidizing aspiration sheath catheter may be rigidized using positive pressure; alternatively in some examples the rigidizing aspiration sheath catheter may be rigidized using negative pressure. In some examples the rigidizing aspiration sheath catheter may be rigidized by both negative and positive pressure.

Also described herein are aspiration catheters (which may be rigidizing or non-rigidizing), a suction line, including an in-line vacuum activation valve and a clot capture chamber. All of some of these components may be used together, e.g., as part of system, or each of them may be used separately and may be configured to include elements of particular use in removing clot material.

The apparatuses and methods described herein may be used for aspiration of clot material from any region of the vasculature, including (but not limited to) pulmonary embolism, peripheral (e.g., arterial embolism), central (e.g., cerebral thrombus), etc., including treatment of stroke.

The aspiration of clots can involve removal of blood. The blood must be captured, and in some cases the blood must be returned to the patient. The present invention contemplates methods to capture, store and return blood to the patient. These include the use of filters and containers that are capable of separating blood in a sterile field. The system also anticipates the use of standard cardiotomy reservoirs. Cardiotomy reservoirs are indicated for use in cardiopulmonary bypass circuits during surgery. Intended uses include an air-fluid separation chamber, a temporary storage reservoir for priming solutions, filtration of particulate materials (clots, blood cell aggregates, etc.). Design accommodations in the present system would allow these reservoirs to be uniquely adapted to an aspiration embolectomy system.

In general, the methods and apparatuses described herein may provide superior control of the catheter or catheters within the vasculature by using dynamic rigidization. For example, a rigidizing aspiration sheath catheter may be positioned at or near a target location within the body in the flexible configuration (or by shifting between a rigid and flexible state), but may be rigidized to provide stability to other components that may pass through and/or extend from the apparatus. These methods and apparatuses may be used with a guidewire, e.g., to help position and/or navigate the catheter, however it may be preferable to operate these apparatuses without the use of a guidewire. Thus, these methods and apparatuses may be used without a guidewire, as the position of the catheter may be maintained by rigidizing the rigidizing aspiration sheath catheter. Thus, the rigidizing aspiration sheath catheter may be both a conduit for delivery and/or removal of material within the vasculature and may provide a stable platform for performing all or part of the procedure, including clot material and further navigation.

The systems described herein, which may include the rigidizing aspiration sheath catheter, one or more additional catheters (e.g., aspiration catheters), rigidizing pressure source (e.g., insufflator, etc.), and vacuum line components (e.g., vacuum activation valve, clot capture chamber, etc.) may be configured to remain within the sterile field during a medical procedure using these apparatuses. For example, the rigidizing aspiration sheath catheter may be positioned within the body and used for multiple introductions and removal steps, typically without requiring the use of a guidewire. Aspiration without a guidewire enables larger luminal area (and therefore greater suction), it reduces guidewire risks, and enables for smoother clot transit, because the clot does not need to shear relative to the wire. This may allow the system to be operated within the limited sterile filed during the entire procedure.

Because a rigidized cannula creates a fulcrum at its tip, the behavior of otherwise flexible aspiration catheters or other flexible instruments is altered accordingly. Unlike a standard system that must be navigated and controlled from the access site (e.g. femoral artery), a device that is manipulated within a rigidized cannula has different control dynamics. The navigable portion of a flexible device is shortened, leading to improved local control. In addition, because of the repositioned fulcrum, the tip of the rigidizing cannula can be used as a pivot point. In this case, a tensioning member within the flexible device can be tensioned to cause the flexible device to flex at the fulcrum. Since the flexible device can be moved in and out of the device, the pivot point moves accordingly. A tensioning member within the flexible device can alter the orientation of the tip thereby improving its navigability.

A tensioning member in the flexible instrument is controlled by an actuator located near the handle of the instrument.

In general, these methods and apparatuses may be used as part of any appropriate medical procedure, in particular vascular procedures, such as, but not limited to, clot removal from the pulmonary vasculature, neurovasculature, coronary vasculature, or peripheral vasculature.

For example, described herein are apparatuses including one or more rigidizing aspiration sheath catheter. A rigidizing aspiration sheath catheter may include: an elongate flexible body comprising a plurality of layers, including a rigidizing layer and a compression layer (e.g., a bladder layer) that is configured to transition the rigidizing layer between a flexible state and a rigid state, and a lumen extending through the elongate flexible body; and a hemostasis valve region extending proximally from the elongate flexible body, the hemostasis valve region comprising a housing having a central bore that is continuous with the lumen, and comprising an annular seal within the central bore and at least one actuator movable coupled to the housing and configured to open and close the annular seal or to seal around a device positioned within the central bore, wherein the at least one actuator is sized and shaped to be used by one hand of a user.

The elongate flexible body may be configured to rigidize along all or portion of the length of the body. The elongate flexible body may generally be an elongate tubular body, and may be any appropriate length (e.g., between 0.5 m and 2 m, between 0.7 m and 1.5 m, etc.), and any appropriate outer diameter (e.g., between 4 French and 35 French, between 10 French and 28 French, between 20 French and 30 French, etc.). The elongate flexible body may be coated inside or outside with a lubricious (e.g., hydrophilic) coating or a hydrophobic coating (for example, parylene).

As will be described in greater detail here, the elongate flexible body may include a plurality of layers, including one or more support layers. For example, the plurality of layers may comprise an inner and/or outer wound coil layer. The coil wound layer may support the rigidizing layer(s) and/or may be configured to withstand the application of suction through the lumen.

Any of the rigidizing apparatuses, including the rigidizing aspiration sheath catheters and (in some examples) the aspiration catheters, described herein may include rigidizing layers or regions that engage with a compression layer (which may be or may include a bladder, e.g., “bladder layer”) that applies force to the rigidizing layer to rigidize the rigidizing layer or in some cases to de-rigidize (e.g., release from rigidization) the rigidizing layer. In some examples, these rigidizing apparatuses may include a rigidizing layer that could include a braid, knit, woven, chopped segments, randomly distributed or randomly oriented filaments or strands, engagers, links, scales, plates, segments, particles, granules, crossing filaments, or other materials forming the rigidizing layer. For example, the rigidizing layer may comprise multiple strand lengths or strand segments that cross over each other (e.g., as part of a braid, knit, woven, etc.); the compression layer may apply force to drive the crossing strand lengths or strand segments against each other. Although many of the examples shown herein are braids, any of these apparatuses may instead or in addition include a general rigidizing layer comprising crossing strand lengths or strand segments. The examples of rigidizing apparatuses described herein may use positive pressure and/or negative pressure (vacuum) to selectively and controllable rigidize. The examples of rigidizing apparatuses described herein may use pressure (positive pressure) together with negative pressure (vacuum), for example, by ‘pushing’ with positive pressure on one side of a membrane, and ‘pulling’ with vacuum on the other side of a bladder.

In general, the rigidizing aspiration sheath catheters described herein may include a lumen, which may be central or may be offset, that is configured to allow passage of a guidewire, a dilator, contrast, a divided lumen (‘double lumen’), and one or more additional devices (e.g., a suction catheter). In some examples the rigidizing aspiration sheath catheters described herein include a lumen that includes a lubricious internal coating or layer, and/or is formed of a lubricous material, such as a hydrophilic material. The lumen of the rigidizing aspiration sheath catheters may be any appropriate size (e.g., inner diameter). For example, the rigidizing aspiration sheath catheters may have an inner diameter that is between about 4 French and 35 French, between about 10 French and 20 French, between about 12 French and 18 French, etc. The inner diameter may be substantially uniform along the length of the lumen. In some examples the inner diameter may include one or more narrowing (tapered) regions along the length. For example in some examples the distal end region of the lumen may be conical, cone-shaped, or tapered. The geometric change may be tapered towards a larger diameter, or towards a smaller diameter. This geometric change may be a stable geometry, or it may be a compressible or collapsible geometry, for example, a nitinol-based conical structure that has an overlaid membrane “skin.”

The proximal end of the lumen of the rigidizing aspiration sheath catheter may be continuous with the central bore of the hemostasis valve region. Thus, the hemostasis valve region may generally seal off the entrance into the lumen of the rigidizing aspiration sheath catheter. The hemostasis valve region may be integral with the proximal end of the rigidizing aspiration sheath catheter and may be adapted for use with the rigidizing aspiration sheath catheter. For example, the hemostasis valve region may operate as a handle region the proximal end of the rigidizing aspiration sheath catheter and may include one or more controls for operating the rigidizing aspiration sheath catheter, allowing easy and intuitive operation of the rigidizing catheter. For example, the one or more actuators (and preferably a pair of actuators) for opening and/or closing the annular seal of the hemostasis valve region may be on the housing of the hemostasis valve region. The one or more actuators may be levers, and may be sized for hand-held operation. One or more controls for controlling rigidization, e.g., turning on/off pressure for rigidizing the elongate flexible body by applying pressure (positive or negative pressure) to the compression (e.g., bladder) layer, may be located on or adjacent to the housing of the hemostasis valve region. In some examples, the rigidizing aspiration sheath catheter may include a pressure inlet coupled to the housing that is configured to receive positive or negative pressure to transition the rigidizing layer between the flexible state and the rigid state. In some examples the hemostasis valve region comprises a bladder adapter configured to allow communication between a pressure source and the bladder layer to control rigidization of the elongate flexible body.

As mentioned, any of the rigidizing aspiration sheath catheters described herein may be configured to apply suction through the lumen of the rigidizing aspiration sheath catheter. In some cases the rigidizing aspiration sheath catheter may include a mating attachment connector proximal to the hemostasis valve region and configured to lockingly engage with a mating attachment of a suction line. This mating attachment connector may be a universal connector that is similar, or identical to, the mating attachment connector of the aspiration catheter. Thus, the same suction line may be attached (swapped between) the aspiration catheter and the rigidizing aspiration sheath catheter. Alternatively in some examples, the suction may be applied to both the rigidizing aspiration sheath catheter and an aspiration catheter within the lumen of the rigidizing aspiration sheath catheter (e.g., around the aspiration catheter).

Any of the rigidizing aspiration sheath catheters described herein may include an atraumatic tip at the distal end of the rigidizing aspiration sheath catheter. For example, the distal end (tip) region) may be formed of a relatively soft (e.g., low durometer) material and may be rounded.

As mentioned, the rigidizing aspiration sheath catheters described herein may be configured to rigidize by the application of pressure. For example, the application of positive or negative (vacuum) pressure. In some examples the elongate flexible body may be configured to transition from the flexible state to the rigid state upon application of positive pressure. In some examples the elongate flexible body is configured to transition from the flexible state to the rigid state upon application of negative pressure. In some examples the elongate flexible body is configured to transition from the flexible state to the rigid state upon application of negative pressure to one side of a bladder simultaneous with the application of positive pressure to the other side of a bladder.

For example, a rigidizing aspiration sheath catheter may include: an elongate flexible body comprising a plurality of layers, including a rigidizing layer and a compression layer that is configured to transition the rigidizing layer between a flexible state and a rigid state, and a lumen extending through the elongate flexible body; and a hemostasis valve region extending proximally from the elongate flexible body, the hemostasis valve region comprising a housing having a central bore that is continuous with the lumen, and comprising an annular seal within the central bore and at least one actuator movable coupled to the housing and configured to open and close the annular seal or to seal around a device positioned within the central bore, wherein the at least one actuator is sized and shaped to be used by one hand of a user; and a mating attachment connector proximal to the hemostasis valve region and configured to lockingly engage with a mating attachment of a vacuum line to apply suction within the lumen.

Also described herein are systems including any of these rigidizing aspiration sheath catheters. Each of the components of these systems may be used separately or in combination. These systems may include multiple versions of any of these components. For example a cardiovascular aspiration system (e.g., a system for aspiration of clot material) may include: any of the rigidizing aspiration sheath catheters described herein, any of the aspiration catheters described herein, and any of the suction lines (including all or some of the suction line components described herein).

As mentioned, the rigidizing aspiration sheath catheter may include: an elongate flexible body comprising a plurality of layers, including a rigidizing layer and compression layer that is configured to transition the rigidizing layer between a flexible state and a rigid state, and a lumen extending through the elongate flexible body, and a hemostasis valve region at a proximal end of the elongate flexible body, and comprising a housing having a central bore that is continuous with the lumen, and comprising a first annular seal within the central bore that is continuous with the lumen, and comprising an annular seal within the central bore and at least one actuator movable coupled to the housing and configured to open and close the annular seal or to seal around a device positioned within the central bore, wherein the at least one actuator is sized and shaped to be used by one hand of a user.

An aspiration catheter may be configured to be inserted through the central bore and into the lumen of the rigidizing aspiration sheath catheter, the aspiration catheter comprising a mating attachment connector at a proximal end. The aspiration catheter may be non-rigidizing. It may be of particularly high flexibility, in that it is engineered to operate within a rigidizing conduit. The region that extends beyond a rigidized catheter may therefore be particularly navigable, in a manner that would not be possible should be exist by itself and without the assistance of a dynamically rigidizing conduit. The aspiration catheter may be rigidizing (e.g., may include a plurality of layers, including a rigidizing layer and a compression (e.g., bladder) layer that is configured to transition the rigidizing layer between a flexible state and a rigid state). In some examples the aspiration catheter is steerable. For example, the aspiration catheter may include one or more actuating steering members. The actuating steering members may be any appropriate steering member, including mechanical steering members (e.g., one or more tendons, cables, wires, etc., actuators, etc.), pneumatic steering members, magnetic steering members, thermal steering members (e.g., using a shape memory alloy or shape memory polymers, etc.). Although the examples described herein include primarily actuating steering members comprising one or more cables, any appropriate actuating steering member may be used in any of these apparatuses and methods.

A vacuum line may include: a mating attachment at a distal end of vacuum line configured to lockingly engage with the mating attachment connector of the aspiration catheter. As mentioned, the mating attachment may be a universal attachment for coupling with one or more of the catheters of the system. For example, the mating attachment may include a bayonet-type attachment as well as one or more finger-grip regions enhancing the ease of use. The vacuum line may include vacuum tubing. Any of these apparatus may include a vacuum activation valve. This apparatus may be hand-triggered, comprising a handle and configured to open or close the vacuum line, wherein the hand-triggered vacuum activation valve is connected in-line with the mating attachment, wherein a proximal end of the vacuum line is further configured to couple to vacuum pump and blood collection chamber. This valve may be a roller mechanism that pinches the vacuum tubing locally.

In some examples the system includes a clot capture chamber that may be connected in-line with hand-triggered vacuum activation valve and may include a visualization chamber mounted above an exit port, wherein the exit port is connected to the proximal end region of the vacuum line. The clot capture chamber may include a removable tray within the visualization chamber. The removable tray may be sized to conform to an inner perimeter of the clot capture container. In some examples the clot capture chamber comprises a transparent lid, which may include one or more tabs. The tray may have a mesh size such that blood passes through, but clot does not. For example, the mesh may have a mesh size of between about 0.037 mm and about 6 mm (e.g., between about 0.05 mm and about 5 mm, between about 0.1 mm and about 4 mm, between about 0.2 mm and 3 mm, between about 0.3 mm and 2.5 mm, between about 0.5 mm and about 4 mm, between about 0.75 mm and about 4 mm, etc.).

As mentioned, any of these systems may include an aspiration catheter configured to be inserted through the central bore and into the lumen of the rigidizing aspiration sheath catheter, the aspiration catheter comprising a second mating attachment connector, wherein the mating attachment at the distal end of vacuum line is configured to lockingly engage with the second mating attachment connector or the first mating attachment connector. The aspiration catheter may comprise an aspiration lumen extending therethrough and an aspiration catheter hemostasis valve region at a proximal end.

For example, a system for clot aspiration may include a rigidizing aspiration sheath catheter comprising: an elongate flexible body comprising a plurality of layers, including a rigidizing layer and bladder layer configured to transition the rigidizing layer between a flexible state and a rigid state, and a lumen extending through the elongate flexible body; and a hemostasis valve region at a proximal end of the elongate flexible body, and comprising a housing having a central bore that is continuous with the lumen of the elongate flexible body, and comprising a first annular seal within the central bore that is configured to seal around a device positioned within the central bore, and at least one first actuator movable coupled to the housing and configured to open and close the annular seal, wherein the at least one or more actuators is sized and shaped to be used by one hand of a user, and a first mating attachment connector; an aspiration catheter configured to be inserted through the central bore and into the lumen of the rigidizing aspiration sheath catheter, the aspiration catheter comprising: an elongate aspiration body including an aspiration lumen extending therethrough; a hemostasis valve region at a proximal end of the aspiration catheter, the hemostasis valve region comprising a housing having an aspiration hemostasis valve central bore that is continuous with the aspiration lumen, and an annular seal within the aspiration hemostasis valve central bore configured to seal around a device positioned within the central bore, at least one second actuator movable coupled to the elongate aspiration body, and configured to open and close the annular seal, wherein the at least one second actuator is sized and shaped to be used by one hand of the user, and a second mating attachment connector; and a vacuum line comprising: a mating attachment at a distal end of vacuum line configured to lockingly engage with the first mating attachment connector or the second mating attachment connector; a hand-triggered vacuum activation valve comprising a handle and configured to open or close the vacuum line, wherein the hand-triggered vacuum activation valve is connected in-line with the mating attachment; a clot capture chamber connected in-line with hand-triggered vacuum activation valve, and comprising a visualization chamber mounted above an exit port, wherein the exit port is connected to the proximal end region of the vacuum line; and wherein a proximal end of the vacuum line is further configured to couple to vacuum pump and blood collection chamber.

In general, a method for removing clot material may include: advancing a rigidizing aspiration sheath catheter in a vessel while the rigidizing aspiration sheath catheter is in a flexible state so that a distal end of the rigidizing aspiration sheath catheter is near a clot material within a vessel, wherein the proximal end of the rigidizing aspiration sheath catheter comprises a hemostasis valve portion; transitioning the rigidizing aspiration sheath catheter from the flexible state to a more rigid state; and aspirating through the rigidizing aspiration sheath catheter in the more rigid state to remove the clot material.

For example, described herein are methods of using any of the apparatuses described herein. For example, described herein are methods of removing clot material that may include: advancing a rigidizing aspiration sheath catheter so that a distal end of the rigidizing aspiration sheath catheter is at or near a treatment location, wherein the proximal end of the rigidizing aspiration sheath catheter comprises a hemostasis valve portion; transitioning the rigidizing aspiration sheath catheter from a flexible state to a rigid state; inserting an aspiration catheter through a hemostasis valve portion of the rigidizing aspiration sheath catheter, wherein the aspiration catheter is coupled to a vacuum line; extending the aspiration catheter distally out of the rigidizing aspiration sheath catheter; and activating a handle of a hand-triggered activation valve that is in-line with the vacuum line to aspirate through the aspiration catheter.

Any of these methods may include observing a clot material aspirated through the aspiration catheter within a window of a clot capture chamber connected in-line with hand-triggered vacuum activation valve.

In general, these methods may include applying suction through the rigidizing aspiration sheath catheter instead of, or in addition to, applying suction through the aspiration catheter. For example, any of these methods may include coupling the vacuum line to the proximal end of the rigidizing aspiration sheath catheter and activating the handle of the hand-triggered activation valve that is in-line with the vacuum line to aspirate through the rigidizing aspiration sheath catheter.

As mentioned, these apparatuses may be used with or without a guidewire, either for the entire procedure or for the portion of the procedure following initially placing the rigidizing aspiration sheath catheter (e.g., rigidizing overtube). For examples, advancing the rigidizing aspiration sheath catheter may include advancing the rigidizing aspiration sheath catheter without the use of a guidewire. In some examples advancing the rigidizing aspiration sheath catheter comprises advancing the aspiration catheter distally relative to the rigidizing aspiration sheath catheter in the rigid state and steering a distal end of the aspiration catheter. The method may further include advancing the rigidizing aspiration sheath catheter in the flexible state over the aspiration catheter and rigidizing the rigidizing aspiration sheath catheter. In some examples advancing the rigidizing aspiration sheath catheter comprises advancing the aspiration sheath catheter with an obturator within a lumen of the rigidizing aspiration sheath catheter.

Any of these apparatuses and methods may include hemostasis valves built into the rigidizing aspiration sheath catheter. For example, a method may include actuating an actuator of the hemostasis valve portion to allow insertion of the aspiration catheter through its central bore.

For example, described herein are rigidizing aspiration sheath catheter apparatus that include a rigidizing elongate body (e.g., a pressure-rigidizing elongate body) that is integrated with a hemostasis valve at the proximal end. As mentioned, an apparatus may include: an elongate body comprising lumen extending therethrough, the elongate body further comprising a rigidizing layer and a bladder layer that are configured to transition the elongate body between a flexible state and a rigid state by the application of pressure; and a hemostasis valve region extending proximally from the elongate flexible body, the hemostasis valve region comprising a housing having a central bore that is continuous with the lumen, an annular seal within the central bore, and at least one actuator movably coupled to the housing and configured to open and close the annular seal or to seal around a device positioned within the central bore.

Any of these apparatuses may be systems that also include one or more aspiration catheters. The aspiration catheter may also include an integrated hemostasis region (e.g., at a proximal end). The proximal ends of the rigidizing aspiration sheath catheters and the aspiration catheters may be adapted to interchangeably engage with a suction (aspiration) line. Any of the rigidizing aspiration sheath catheters may be steerable or may be steered by using an obturator and/or guidewire. In some cases the rigidizing aspiration sheath catheter include one or more distal steering regions that may be steered from the proximal end, e.g., by pulling on a tendon. In some cases the obturator configured to fit into the rigidizing aspiration sheath catheter may be steerable. In some cases the obturator adapted to fit snugly into the rigidizing aspiration sheath catheter may adapt the rigidizing aspiration sheath catheter for use with a guidewire (e.g., the obturator may include a guidewire lumen).

The methods described herein may be methods for treatment of any cardiovascular disorder, including, but not limited to removing clot material from an artery or a vein, including but not limited to the neurovasculature, the coronary vasculature, the peripheral vasculature, or the pulmonary vasculature, including the pulmonary artery. For example, advancing the rigidizing aspiration sheath catheter to the treatment location may include advancing a distal end of the rigidizing catheter to a pulmonary artery of a patient.

Although in general, these methods and apparatuses describe the use of a rigidizing aspiration sheath catheter (rigidizing overtube) and one or more non-rigidizing aspiration catheters that may extend from the rigidizing aspiration sheath catheter, any of these methods and apparatuses may include a rigidizing (e.g., pressure rigidizing) aspiration catheter. Thus, in some cases both the rigidizing aspiration sheath catheter and the aspiration catheter may be rigidizing by the application of pressure (positive and/or negative pressure).

In general, these methods and apparatuses may hold the rigidizing overtube in a rigid state while applying suction (aspiration) through one or both of the rigidizing overtube and/or an aspiration catheter. This may be particularly, and surprisingly, advantageous, as it may prevent movement of the rigidizing aspiration sheath catheter (e.g., rigidizing overtube) within the lumen of the vessel, even when the distal tip of the rigidizing aspiration sheath catheter is near the vessel wall, and even at relatively high suction (e.g., flow rate). This may prevent latching of the rigidizing aspiration sheath catheter to the wall. Rigidizing the rigidizing aspiration sheath catheter may also prevent inadvertent movement of the aspiration catheter that may otherwise occur within the vessel when aspiration is applied.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows one example of a system including a rigidizing aspiration sheath catheter apparatus as described herein.

FIGS. 1B-1G show examples of a system and components of these systems as described herein.

FIG. 2A shows an example of a rigidizing device.

FIGS. 2B-2C show exemplary rigidized shapes of a rigidizing device.

FIGS. 3A-3B show an example of a portion of a vacuum rigidizing apparatus as described herein. FIG. 3A shows a section through the exemplary vacuum rigidizing member of the apparatus. FIG. 3B shows an enlarged view of a portion of the section, illustrating the arrangement of layers in the un-rigidized configuration.

FIGS. 3C-3F show an example of a portion of a vacuum rigidizing apparatus having multiple rigidizing layers as described herein. FIG. 3C shows a perspective view of the vacuum rigidizing member with the outer layer removed (showing the outermost rigidizing layer). FIG. 3D is an enlarged view of a portion of FIG. 3C. FIG. 3E shows a longitudinal section though the vacuum rigidizing member of FIG. 3C. FIG. 3F is a cross-section through the rigidizing member of FIG. 3C.

FIGS. 4A-4B show an exemplary pressure rigidizing device.

FIG. 5 shows a rigidizing device with a distal end section.

FIG. 6 shows a rigidizing device with a distal end section having a plurality of actively controlled linkages.

FIG. 7A schematically illustrates an example of a section through a cross-section on one example of a proximal end of a rigidizing aspiration sheath catheter (also referred to herein as simply a rigidizing catheter).

FIGS. 7B-7D show various views of an embodiment of a hemostasis valve for use with rigidizing aspiration system.

FIG. 8 shows an embodiment of an aspiration catheter handle.

FIGS. 9A-9D show various views of a control valve, e.g., a hand-activated vacuum activation valve.

FIG. 9E illustrates an example of a control valve, similar to the vacuum control valve of FIGS. 9A-9D, including a retainer to retain the handle in an “open” configuration for storage.

FIGS. 10A-10H show various views of a clot capture container.

FIGS. 11A and 11B show a traditional catheter and a rigidizing catheter, respectively, navigating to the pulmonary artery.

FIGS. 12A-12B illustrate an example of a system including a rigidizing aspiration sheath catheter configured with a blood return circuit, including a blood filter and blood bag as described herein.

FIGS. 13A-13B illustrate an example of a system including a rigidizing aspiration sheath catheter configured with a blood return circuit without a blood bag, as described herein.

FIGS. 14A-14B illustrate an example of a system including a rigidizing aspiration sheath catheter configured with a blood return circuit having separate access and return sites on the body, as described herein.

FIGS. 15A-15B illustrate an example of a system including a rigidizing aspiration sheath catheter configured with a blood return circuit including a blood bag, as described herein.

FIGS. 16A-16B illustrate an example of a system including a rigidizing aspiration sheath catheter including a blood return circuit using a syringe, as described herein.

FIG. 16C is an example of a portion of a tubing assembly that may be used with a blood return circuit as described herein.

FIGS. 17A-17B illustrate an example of a system including a rigidizing aspiration sheath catheter. FIG. 17A shows a schematic illustration of the system. FIG. 17B illustrates the system of FIG. 17A including the optional blood return circuit portion.

FIG. 18 illustrates an example of a robotic system including a rigidizing aspiration sheath catheter as described herein.

FIGS. 19A-19B illustrate an example of a clot capture container that is configured to reduce hemolysis.

FIGS. 20A-20B illustrate an example of a system including a venting control (e.g., venting button) that may reduce hemolysis.

### DETAILED DESCRIPTION

Described herein are methods and apparatuses (e.g., devices, systems, etc.) for cardiovascular treatment. These methods and apparatuses may be configured for removal of clot material (e.g., for clot capture) by aspiration, and may include one or more rigidizing (e.g., dynamically rigidizing) catheters that can provide a platform for the aspiration system. Using a rigidizing system to navigate to locations within the vasculature, for example deep within the vasculature, can advantageously allow for creation of a stable pathway to the treatment site through which aspiration can be performed. Using a stable pathway can advantageously allow for enhanced kinematics of catheter based instruments used during interventional procedures. For example, in some embodiments, the system allows for 1:1 control of catheter-based instruments. Additionally, a stable pathway reduces the need for repeated navigation to the treatment site and repeated use of contrast during visualization of the navigation. These advantages of using a rigidizing system platform simplify procedures, greatly improve outcomes, and increase patient safety.

In general, these method and apparatuses may include a rigidizing (e.g., dynamically rigidizing) aspiration sheath catheter that may include an elongate flexible body having a plurality of layers, including one or more rigidizing layer and a compression layer (e.g., a bladder layer) configured to transition the rigidizing layer between a flexible state and a rigid state. The rigidizing aspiration sheath catheter may include a lumen extending through the elongate flexible body and an integrated hemostasis valve. The integrated hemostasis valve may be part of a hemostasis valve region at a proximal end of the elongate flexible body.

The dynamically rigidizing catheters described herein may be controllable switched between a highly flexible state and one or more (or a continuously increasing) stiff or rigid state(s). In some examples the rigidizing catheter may be configured so that the flexible states is the resting state (e.g., without a pressure differential being applied). In general, the flexible state may be a low resting energy state. That is, in its flexible state the catheter may exert very low forces on the anatomy. In its rigid state(s) the catheter may exert low forces on the anatomy, and when the catheter is released (either intentionally or accidentally) it may still have a low potential energy. Thus, the catheter may avoid or prevent movement recoil (e.g., “kick”, which may occur when transitioning to applying suction), and may stay in the same position. This may be particularly beneficial as compared to non-rigidizing devices that may have higher energy states (sometimes much higher), and that apply a recoil force that can injure the patient when released (either intentionally or accidentally).

These method and apparatuses may also include an aspiration catheter that may be inserted into the rigidizing aspiration sheath catheter, which may also include a hemostasis valve region. The aspiration catheter may be steerable (e.g., may include an actuating steering member). In some examples the aspiration catheter may be rigidizing as well.

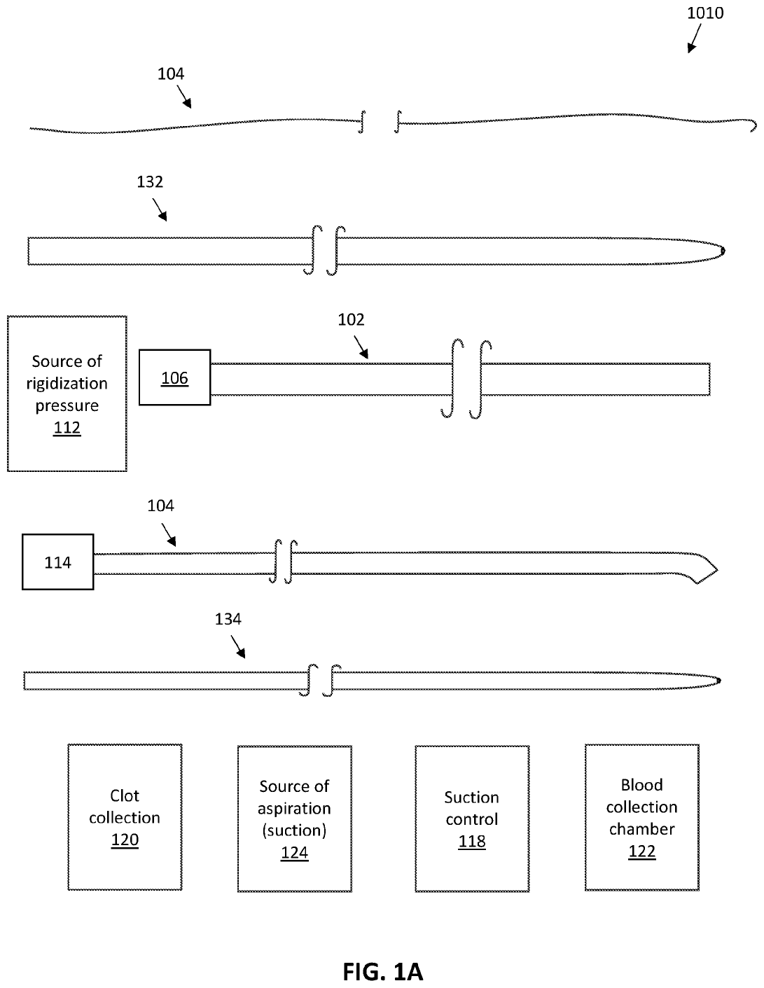

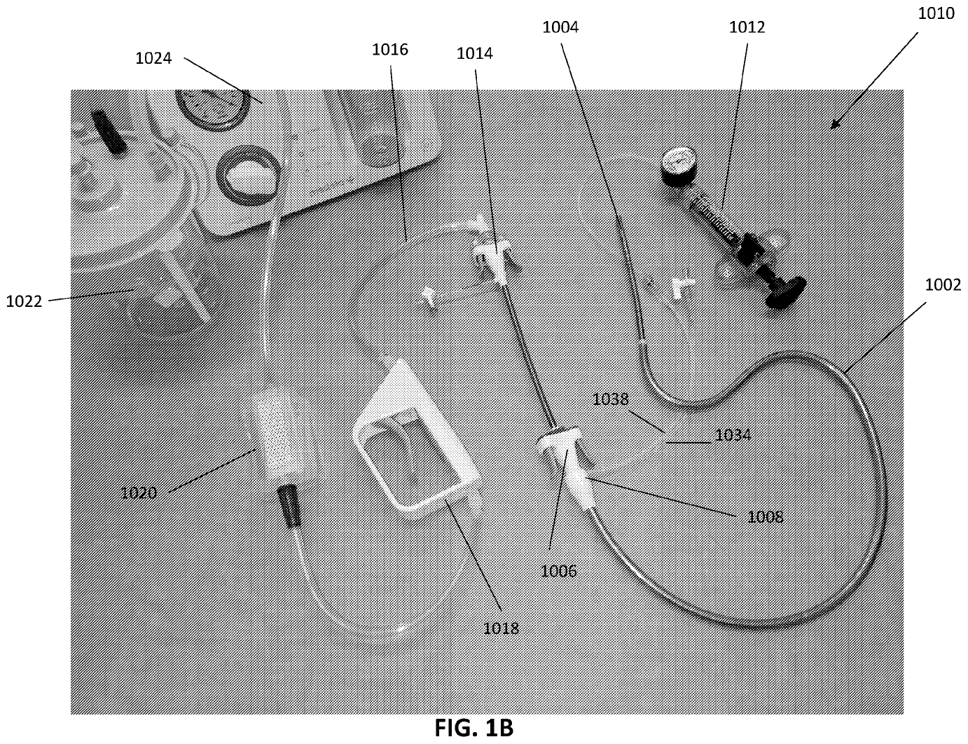

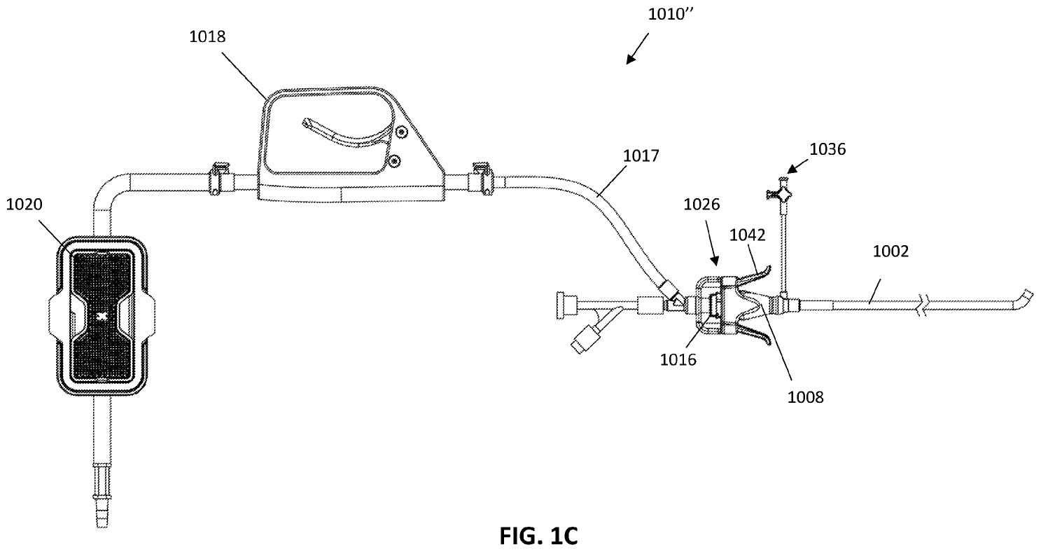

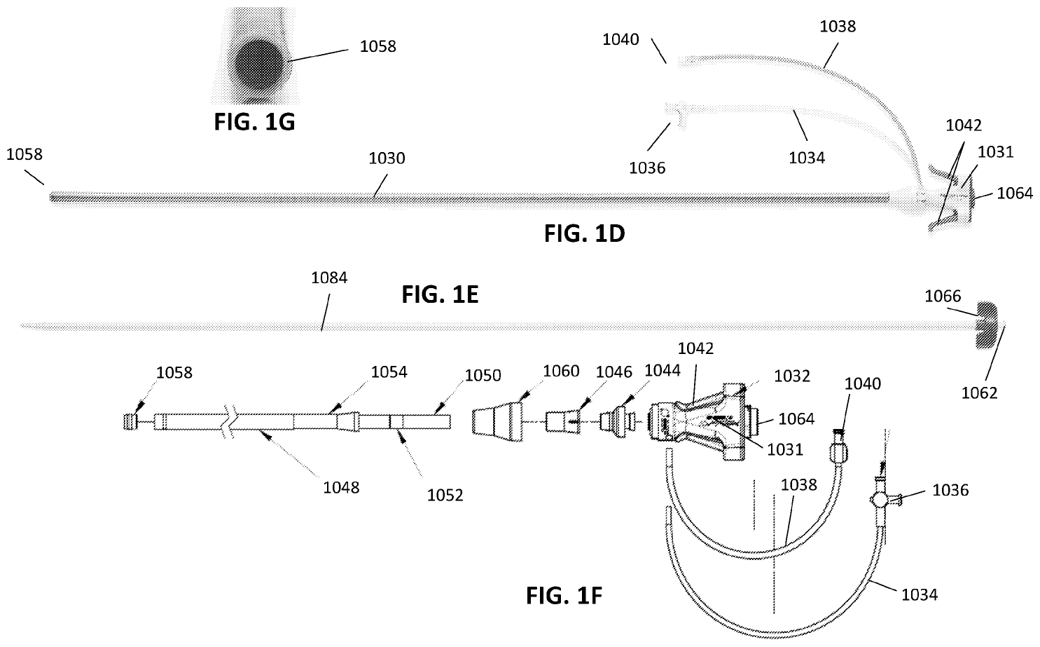

In some examples the method and apparatuses described herein may include a vacuum line with one or more of: a hand-triggered vacuum activation valve and/or a clot capture chamber for visualizing and removing clot material aspirated by the system. FIG. 1A schematically illustrates one example of an apparatus (e.g., system) including at least a rigidizing aspiration sheath catheter 102 that including a proximal region configured as a hemostasis valve region 106. The rigidizing aspiration sheath catheter is generally configured to be changed between a flexible state (flexible configuration) and a less flexible, e.g., rigid, state (rigid configuration). Any appropriate structure for rigidizing may be used, including in particular a layered structure that is rigidized by the application of positive and/or negative pressure. For example, these apparatuses may be configured as a rigidizing aspiration sheath catheter configured to couple to a source of positive and/or negative pressure 112, e.g., through a port or inlet on the proximal end (which may be part of the hemostasis valve region or separate from it) to control the rigidity of the rigidizing aspiration sheath catheter. In some examples the rigidizing aspiration sheath catheter apparatus has an elongate body comprising lumen extending therethrough. The elongate body may include layers, such as a rigidizing layer and a bladder layer that are configured to transition the elongate body between a flexible state and a rigid state by the application of pressure. As described in greater detail below, the bladder layer (e.g., “bladder”) may be driven against (or allowed to move away from) the rigidizing layer to control the flexibility/stiffness of the elongate body. The rigidizing layer may comprise a plurality of overlapping filament lengths that are free to slide over each other in the more flexible state(s), but may the bladder layer may be driven against the rigidizing layer, and/or against a support layer (e.g., a reinforced layer) on an inner or an outer region of the elongate body, to rigidize the elongate body.

The rigidizing aspiration sheath catheter may be used with one or more obturators 132. In FIG. 1A the obturator may be inserted into the rigidizing aspiration sheath catheter over a guidewire 104 (which may be included with the apparatus 1010 or may be separately provided). This may allow the rigidizing aspiration sheath catheter to be guided over a guidewire positioned in a body vessel. The obturator may be steerable or not. In general, the obturator may be flexible so that the combined obturator and rigidizing aspiration sheath catheter (in the flexible configuration) may readily track over a guidewire. The obturator may be longer than the rigidizing aspiration sheath catheter (e.g., by more than 1 cm (e.g., between 1 cm-20 cm, between 1 cm-30 cm, 1 cm-40 cm, etc. or any number therebetween) to allow tracking while avoiding “fishmouthing” over the rigidizing aspiration sheath catheter distal end opening. The obturator may have an atraumatic tip. The obturator may have regions of different material properties (e.g., stiffnesses), such as described in PCTUS2022082141, filed Dec. 21, 2022, and herein incorporated by reference in its entirety.

The apparatus 1010 (e.g., system) in FIG. 1A may also include one or more aspiration catheters 104 that may also include a proximal hemostasis valve region. The aspiration catheter may also be used with an obturator 134 that may be inserted through the aspiration catheter and inserted through the rigidizing aspiration sheath catheter, e.g., in the rigid configuration. In some cases it may be beneficial for the distal tip region of the aspiration catheter to be directional (e.g., bent, curved, etc.) in a fairly rigid bend, to allow for directional aspiration when extended from the rigid rigidizing aspiration sheath catheter. The aspiration catheter may generally be configured to have a relatively high flexibility with a high torquability. The high torquability may allow the apparatus to be steered (directed) within the rigidizing aspiration sheath catheter when extended distally of the distal end of the rigidizing aspiration sheath catheter, e.g., in the rigid configuration.

Other system components may include tubing (suction line) connecting the rigidizing aspiration sheath catheter and/or aspiration catheter to a source of aspiration 124. The rigidizing aspiration sheath catheter and/or aspiration catheter may be connected view a sealing connection to the suction line, which may be connected in-line to a clot collection chamber 120, and/or a suction (e.g., vacuum) activation valve, which may be activated to apply the suction to the rigidizing aspiration sheath catheter and/or aspiration catheter. The apparatus may also include a blood collection chamber 122 before the source of aspiration 123 (e.g., suction pump). These components may be arranged between the rigidizing aspiration sheath catheter and/or aspiration catheter and the source of aspiration in any appropriate order.

For example, FIG. 1B illustrates an example of an apparatus (e.g., a system) for clot aspiration including these components. In FIG. 1B the system 1010′ includes a rigidizing aspiration sheath catheter 1002 that is shown coupled to an insufflator 1012 to control transitioning between a rigid state and a flexible state. The aspiration system 1010′ also include an aspiration catheter 1004. The dynamically rigidizing aspiration sheath catheter 1002 includes a hemostatic valve region 1006. The hemostatic valve region includes a connection 1008 to a pressure source (e.g., insufflator 1012). The aspiration catheter 1004 extends proximally from the hemostatic valve region to an aspiration catheter handle 1014. Through the aspiration catheter handle, the aspiration catheter comprises a connection to a tube or other elongate element 1016 that connects to a vacuum activation valve 1018. The tube 1016 extends proximally to a clot capture chamber 1020. A vacuum pump 1024 is positioned at a proximal portion of the aspiration lumen 1016 and blood collection container 1022.

FIG. 1C shows another example of a portion of a system 1010″ as described herein, including an aspiration catheter 1002 with an integrated hemostatic valve 1026 and a flush port 1036. The aspiration catheter is shown locking coupled to a mating attachment 1016 at a distal end of vacuum line. The mating attachment is configured to couple to a mating attachment connector on the distal end of the aspiration catheter for making a quick connection to the suction line 1017. A hand-triggered vacuum activation valve 1018 is shown connected in-line with the vacuum line and may be easily used to turn on/off suction through the apparatus. The vacuum line is also connected to a clot capture chamber 1020, described in greater detail below.

FIGS. 1D-1G show embodiments of components of an aspiration system (e.g., like those shown in FIG. 1B). Referring now to FIGS. 1D and 1F, the system may include a rigidizing aspiration sheath catheter 1030 including a hemostatic seal region 1031 at a proximal end. The hemostatic seal region 1032 comprises a body 1031. The seal in this example also includes a tube or elongate element 1034 connecting to a flush port 1036. In some embodiments, the flush port can comprise a luer type adapter. The hemostatic seal region 1032 also includes a tube or elongate element 1038 with a connector 1040 at its proximal end for connection to a pressure source (e.g., an insufflator). The connector may be a luer type connector.

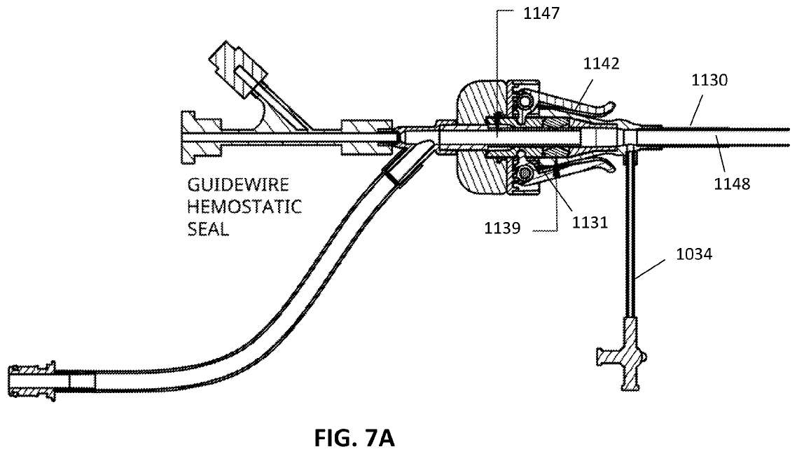

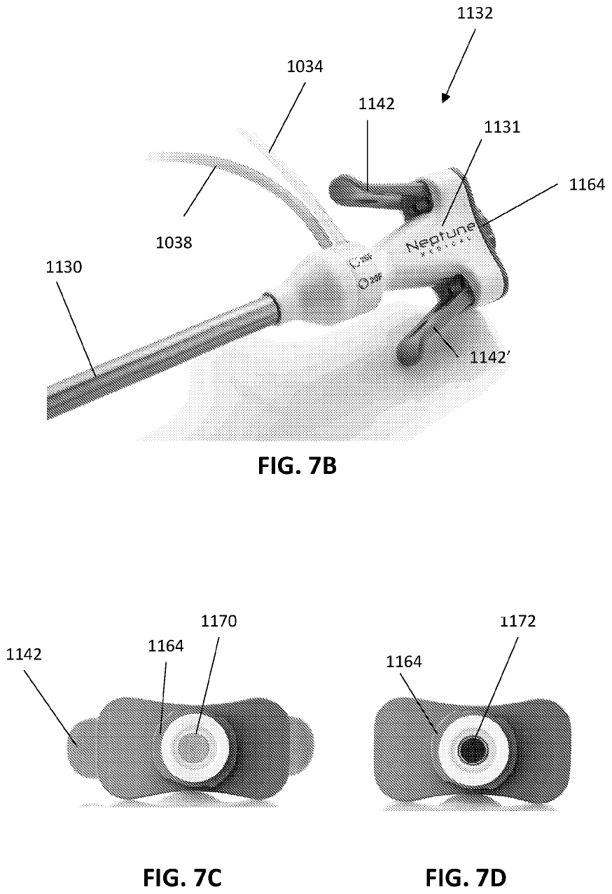

The hemostatic seal region in this example includes a pair of actuators 1042 (shown as levers). Depressing levers 1042 can allow for release of a device (e.g., aspiration catheter) positioned within the rigidizing catheter 1030. When the levers are in their unbiased state, extending outwardly from the body of the seal 1032, the seal valve is closed (shown in FIG. 7D, below).

In FIG. 1F a bladder adapter 1044 for connecting the bladder of the rigidizing aspiration sheath catheter 1030 to the seal 1032 is located a distal end of the seal 1032. This connection allows the seal 1032 to maintain pressurization (e.g., insufflation) of the rigidizing aspiration sheath catheter 1030. Distal to the bladder adapter is an adapter 1046 for connection to an outer layer 1048 of the rigidizing aspiration sheath catheter 1030. A shroud 1060 is located distal to the outer layer adapter 1046, for covering the adapters 1044, 1046 and from which the rigidizing catheter 1030 distally extends.

FIG. 1F further shows an example of the layers of the rigidizing aspiration sheath catheter 1030, including the inner layer 1050, the bladder layer 1052, the rigidizing layer 1054 (e.g., in some examples the rigidizing layer comprises a plurality of filaments that cross over each other, such as, but not limited to, a braid layer, knit layer, woven layer, etc.), and the outer layer 1048. At the distal end of the rigidizing aspiration sheath catheter 1030 is a distal tip 1058.

FIG. 1G shows an end view of the rigidizing catheter shown in FIGS. 1D-1F, in which the rigidizing aspiration sheath catheter includes an atraumatic, distal tip.

In some embodiments, the rigidizing aspiration sheath catheter inner lumen comprises a hydrophilic coating. This coating can help facilitate insertion of an obturator and other devices and accessories.

In some embodiments, an outer surface of the rigidizing aspiration sheath catheter comprises a hydrophobic coating. This type of coating can help facilitate smooth motion through an introducer sheath.

The rigidizing aspiration sheath catheter may have an inner lumen diameter of the lumen of about 0.03-0.6 in. In some embodiments, the rigidizing aspiration sheath catheter inner lumen diameter is about 0.16 in. This inner diameter allows compatibility with a 12F catheter. In some embodiments, the inner lumen diameter is about 0.2-0.3 in. This inner diameter allows compatibility with a 20F catheter. In some embodiments, the rigidizing catheter inner lumen is about 0.34 in. This inner diameter allows compatibility with a 26F catheter.

In some embodiments, the rigidizing catheter outer diameter is about 0.2-0.4 in. In some embodiments, the outer diameter is about 0.23 in. or about 18F. In some embodiments, the outer diameter is about 0.34 in. or about 26F. In some embodiments, the rigidizing catheter as a length of about 70-120 cm (or about 80-115 cm, or about 85-115 cm, etc.). In some embodiments, the rigidizing catheter has a minimum bend radius of about 1-2 in. (or about 1.5 in.).

The system can include an obturator 1084 which can be used during navigation of the rigidizing aspiration sheath catheter 1030. Examples of obturators are described in International Application No. PCT/US2023/062206, filed Feb. 6, 2023, the entirety of which is incorporated by reference herein. The obturator 1084 can comprise a connector 1062 at its proximal end. The obturator 1084 may be configured to be inserted into the rigidizing aspiration sheath catheter 1030 through the hemostatic seal. Once the obturator 1084 is completely inserted within the rigidizing aspiration sheath catheter 1030, the obturator can be rotated to lock it in place with respect to the hemostatic seal. The rotation of connector 1062 relative to a threaded connection 1064 on the hemostatic seal region may create a lock between the mating mechanism 1064 and corresponding mating mechanism 1066 (e.g., thread, bayonet connection, etc.) on the obturator connector 1062. In some embodiments, the connector and obturator are rotated about 90° relative to one another. Other amounts of relative rotation (e.g., about 30-360° are also contemplated).

As described in further details below, the rigidizing aspiration sheath catheter can be transitioned between a flexible and a rigid state upon application of pressure. In the flexible state, the rigidizing aspiration sheath catheter can be navigated (in some examples over a guidewire, though in some examples no guidewire is needed or used) through tortuous anatomy and vasculature. Once the rigidizing catheter has navigated to a desired location, transitioning the rigidizing aspiration sheath catheter to a rigid state preserves the shape of the catheter at the time of rigidization and provides a stable pathway for navigation of other accessories and tools through the catheter, again without requiring a guidewire (or without further use of a guidewire). The ability to transform the rigidizing aspiration sheath provides a number of advantages to the apparatuses described herein that are not realizable with existing apparatuses. In particular, in the flexible configuration the aspiration sheath may conform to the patient's anatomy, whereas a standard catheter is more rigid and therefore exerts a force that forces the anatomy to adapt to it; once converted to a rigid (or semi-rigid) state, the aspiration sheath may prevent or minimize force on the same anatomy, and my support operations from the aspiration sheath.

In some embodiments, the rigidizing aspiration sheath catheter can be configured to be at maximum rigidization about application of 6 atm of positive pressure. In some applications, maximum rigidization can occur with the application of 10, 20, 30, 40, or 50 atm. In some embodiments, the rigidizing catheter can be configured to be flexible upon application of negative pressure. In some embodiments, the rigidizing catheter (rigidizing aspiration sheath catheter) can be configured to be flexible without the application of pressure. Other configurations are also contemplated, as described in further detail below.

Any dynamically rigidizing structure may be used as part of the rigidizing aspiration sheath catheter described herein, or optionally the aspiration catheters. For example, variations of rigidizing aspiration sheath catheters (“rigidizing catheters”) that can be used with the aspiration system described herein can be long, thin, and hollow and can transition quickly from a flexible configuration (i.e., one that is relaxed, limp, or floppy) to a rigid configuration (i.e., one that is stiff and/or holds the shape it is in when it is rigidized). In some examples the rigidizing apparatus may include a plurality of layers (e.g., coiled or reinforced layers, slip layers, rigidizing layers, bladder layers, sealing sheaths, etc.) that can together form the wall of the rigidizing devices, which may be referred to as “layered rigidizing apparatuses.” Unless the context makes clear otherwise, the methods and apparatuses described herein may refer to any appropriate rigidizing device, including layered rigidizing apparatuses. For example, the rigidizing devices (members, apparatuses, etc.) described herein may be rigidized by jamming particles, by phase change, by interlocking components (e.g., cables with discs or cones, etc.) or any other rigidizing mechanism. The rigidizing devices can transition from the flexible configuration to the rigid configuration, for example, by applying a vacuum or pressure to the wall of the rigidizing device or within the wall of the rigidizing device. With the vacuum or pressure removed, the layers can easily shear or move relative to each other, including the rigidizing layer(s). With the vacuum or pressure applied, the layers can transition to a condition in which they exhibit substantially enhanced ability to resist shear, movement, bending, torque and buckling, thereby providing system rigidization. Examples of rigidizing layers that may be rigidized by the application of pressure (positive or negative pressure), may include layers formed of one or more filaments that cross over each other, e.g., in a braid, weave, knit, etc. The rigidizing layer may be organized (e.g., knit, braid, weave, etc.) or may be disorganized (e.g., a mixture of filament fragments of uniform or varying lengths), or some combination of these. The crossing filaments of the rigidizing layer may be adjacent to the bladder layer, which may be driven against the rigidizing layer to rigidize the device. The examples of rigidizing apparatuses described herein may use pressure (positive pressure) and/or negative pressure to selectively and controllably rigidize the apparatus. In general, the method described herein may be used with any appropriate rigidizing apparatus.

The rigidizing (e.g., selectively rigidizing) apparatuses described herein can provide rigidization for a variety of medical applications, including catheters, sheaths, scopes (e.g., endoscopes), wires, overtubes, trocars or laparoscopic instruments. The rigidizing devices can function as a separate add-on device or can be integrated into the body of catheters, sheaths, scopes, wires, or laparoscopic instruments. The devices described herein can also provide rigidization for non-medical structures.

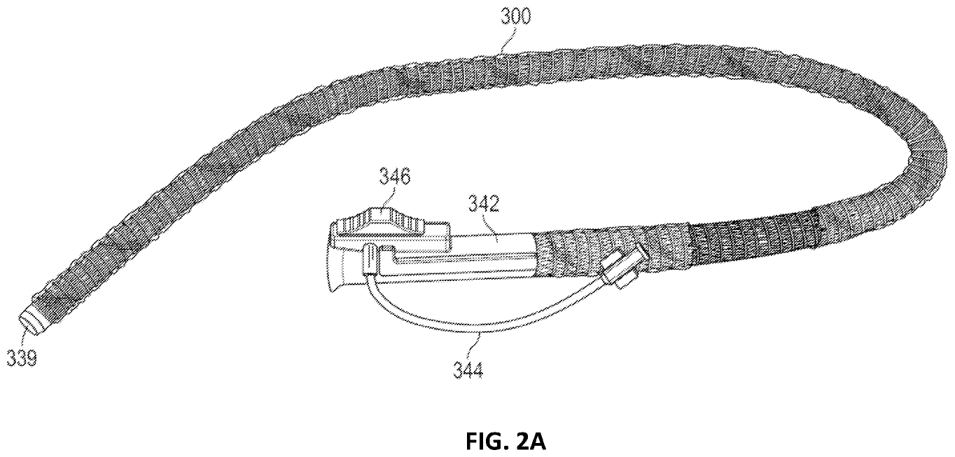

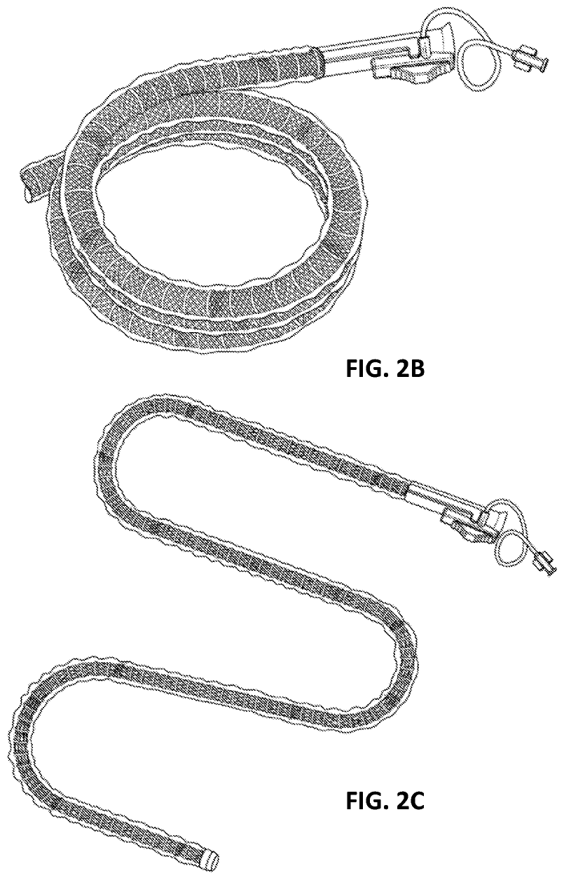

An exemplary rigidizing apparatus is shown in FIG. 2A. The system shown includes a rigidizing device 300 having a wall with a plurality of layers including a rigidizing layer, an outer layer (part of which is cut away in this example to show the braid thereunder), and an inner layer. The system further includes a handle 342 having a vacuum or pressure inlet 344 to supply vacuum or pressure to the rigidizing device 300. A separate flush/perfusion inlet and line (not shown) may also be included, e.g., for applying fluid (e.g., saline, contrast, etc.) through the lumen of the rigidizing overtube. An actuation element 346 can be used to turn the vacuum or pressure on and off to thereby transition the rigidizing device 300 between flexible and rigid configurations. The distal tip 339 of the rigidizing device 300 can be smooth, flexible, and atraumatic to facilitate distal movement of the rigidizing device 300 through the body. Further, the tip 339 can taper from the distal end to the proximal end to further facilitate distal movement of the rigidizing device 300 through the body. In this example, the rigidizing apparatus is configured as an overtube, but other configurations may be used.

Exemplary rigidizing devices in a rigidized configuration are shown in FIGS. 2B and 2C. As the rigidizing device is rigidized, it locks into the shape it was in before vacuum or pressure was applied, i.e., it does not straighten, bend, or otherwise substantially modify its shape (e.g., it may stiffen in a looped configuration as shown in FIG. 2B or in a serpentine shape as shown in FIG. 2C). The air stiffening effect on the inner or outer layers (e.g., made of coil-wound tube) can be a small percentage (e.g., 5%) of the maximum load capability of the rigidizing device in bending, thereby allowing the rigidizing device to resist straightening. Upon release of the vacuum or pressure, braids or strands within the layers forming the device can unlock relative to one another and again move so as to allow bending of the rigidizing device. Again, as the rigidizing device is made more flexible through the release of vacuum or pressure, it does so in the shape it was in before the vacuum or pressure was released, i.e., it does not straighten, bend, or otherwise substantially modify its shape. Thus, the rigidizing devices described herein can transition from a flexible, less-stiff configuration to a rigid configuration of higher stiffness by restricting the motion between the strands of braid (e.g., by applying vacuum or pressure).

The rigidizing apparatuses (e.g., rigidizing aspiration sheath catheter, and in some case aspiration catheters) described herein can toggle between a rigid configuration and a flexible configuration quickly, and in some examples with an indefinite number of transition cycles. In some examples the degree of rigidization (e.g., the stiffness) of the apparatus may also be adjusted, for example, by adjusting the positive pressure (in examples that are rigidized by positive pressure) or vacuum (in examples rigidized by vacuum). As interventional medical devices are made longer and inserted deeper into the human body, and as they are expected to do more exacting therapeutic procedures, there is an increased need for precision and control. Selectively rigidizing devices (including selectively rigidizing overtubes) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein.

The rigidizing aspiration sheath catheters described herein can additionally or alternatively include any of the features described with respect to International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, and International Patent Application No. PCT/US2020/013937 filed on Jan. 16, 2020, titled “DYNAMICALLY RIGIDIZING COMPOSITE MEDICAL STRUCTURES,” the entireties of which are incorporated by reference herein.

The rigidizing devices described herein can be provided in multiple configurations, including different lengths and diameters. In some examples, the rigidizing devices can include working channels (for instance, for allowing the passage of typical endoscopic tools within the body of the rigidizing device), balloons, nested elements, and/or side-loading features.

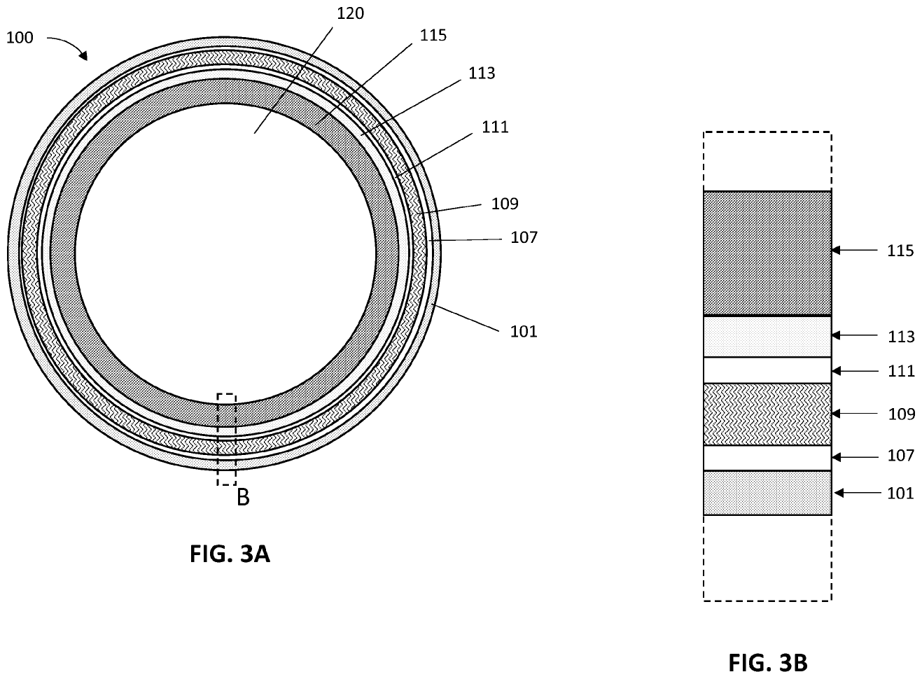

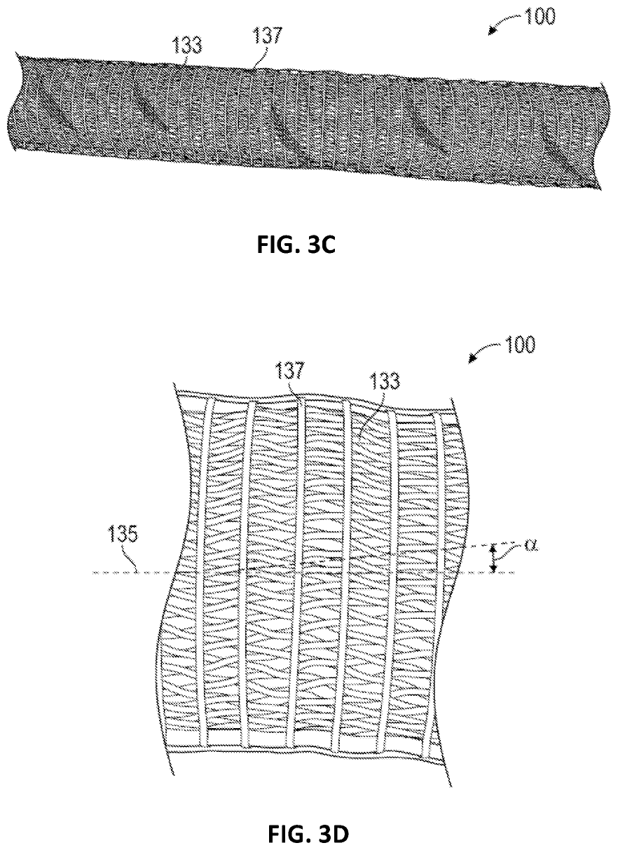

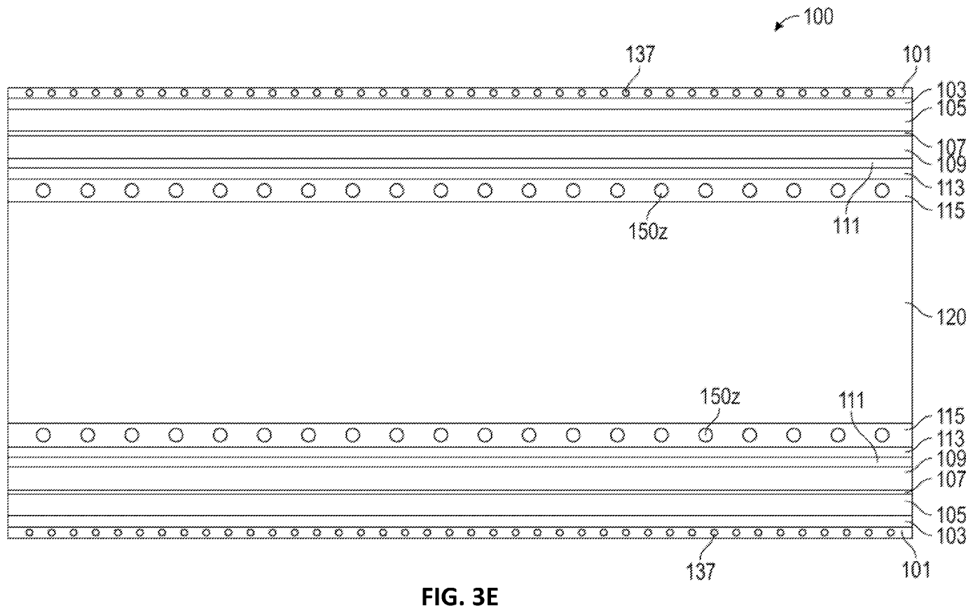

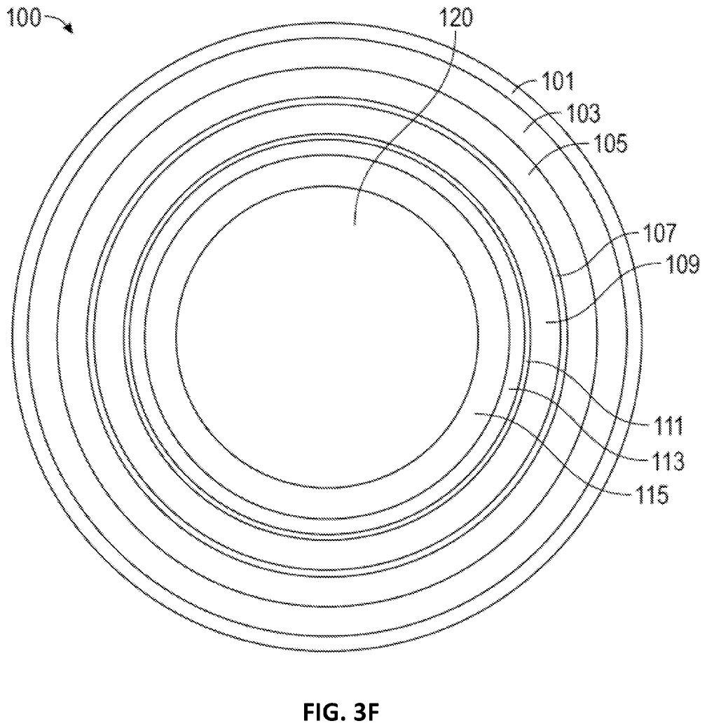

For example, a rigidizing apparatus 100 (also referred to as an apparatus, e.g., system and/or device, including a rigidizable member) may be configured to be rigidized by the application of vacuum, e.g., negative pressure. These apparatuses may generally be formed of layers that are configured to form a laminates structure when negative pressure is applied, so that one or more braided or woven layers may be reversibly fused to a flexible outer layer that is driven against a more rigid inner layer. FIGS. 3A-3B illustrate one example of a section through a rigidizing member of an apparatus (e.g., device, system) that is rigidized by the application of vacuum. FIG. 3B shows an enlarged view of the arrangement of the layers of FIG. 3A in the un-rigidized configuration. In this example, the rigidizable member includes an innermost layer 115 that is configured to provide an inner surface against which the remaining layers can be consolidated (e.g., when vacuum is applied). The innermost layer 115 can include a reinforcement element or coil. The rigidizing member may also include an optional slip layer 113 over (e.g., radially outwards of) the innermost layer. The slip layer may be, e.g., a lubrication, coating and/or powder (e.g., talcum powder) on the outer surface of the inner layer 115 and/or within the gap layer 111. A radial gap layer 111 may separate the slip layer 113 from a rigidizing layer 109 (which in some examples is a knitted, braided or woven layer), providing a space between the rigidizing layer and the slip layer for the braided layer(s) thereover to move within, e.g., when no vacuum is applied; this space or gap may be removed when vacuum is applied, allowing the braided or woven layer(s) to move radially inward upon application of vacuum. A second gap layer 107 may be present between the rigidizing layer 109 and may be similar to layer 111. As will be described in reference to FIGS. 3C-3F, multiple rigidizing layers may be included (e.g., 2, 3 4 or more rigidizing layers may be included) and may be separated by additional gap layers and/or slip layers. The outermost layer 101 can be separated from the rigidizing layer(s) by a gap layer and can be configured to move radially inward when a vacuum is applied to pull down against the rigidizing layer(s) and conform onto the surface(s) thereof. The outermost layer 101 can be soft and atraumatic and can be sealed at both ends to create a vacuum-tight chamber with the innermost layer 115. The outermost layer 101 can be elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be a plastomer. Alternatively, the outermost layer can be a plastic, including, for example, LDPE, nylon, or PEEK.

FIGS. 3C-3F illustrate an example of a tubular rigidizing member of an apparatus 100 that includes multiple rigidizing layers. As in FIGS. 3A-3B, the apparatus includes a tube having a wall formed of a plurality of layers positioned around a lumen 120 (e.g., for placement of an instrument or endoscope therethrough). A vacuum can be supplied between the layers to rigidize the rigidizing device 100. Any of the tubular apparatuses described herein may instead include a solid core forming the inner layer 115.

The innermost layer 115 can be configured to provide an inner surface against which the remaining layers can be consolidated, for example, when a vacuum is applied within the walls of the rigidizing device 100. The structure can be configured to minimize bend force and/or maximize flexibility in the non-vacuum condition. In some examples, the innermost layer 115 can include a reinforcement element 150z or coil within a matrix, as described above. In the example shown in FIG. 3E, the layer 113 over (i.e., radially outwards of) the innermost layer 115 can be a slip layer. The layer 111 can be a radial gap (i.e., a space). The gap layer 111 can provide space for the braided layer(s) thereover to move within (when no vacuum is applied) as well as space within which the braided or woven layers can move radially inward (upon application of vacuum).

The layer 109 can be a first rigidizing layer including braided strands 133 similar to as described elsewhere herein. The rigidizing layer can be, for example, 0.001″ to 0.040″ thick. For example, a rigidizing layer can be 0.001″, 0.003″, 0.005″, 0.010″, 0.015″, 0.020″, 0.025″ or 0.030″ thick. In some examples, as shown in FIG. 3D, the braid can have tensile or hoop fibers 137. Hoop fibers 137 can be spiraled and/or woven into a rigidizing layer. Further, the hoop fibers 137 can be positioned at 2-50, e.g., 20-40 hoops per inch. The hoop fibers 137 can advantageously deliver high compression stiffness (to resist buckling or bowing out) in the radial direction but can remain compliant in the direction of the longitudinal axis 135 of the rigidizing device 100. That is, if compression is applied to the rigidizing device 100, the rigidizing layer 109 will try to expand in diameter as it compresses. The hoop fibers 137 can resist this diametrical expansion and thus resist compression. Accordingly, the hoop fiber 137 can provide a system that is flexible in bending but still resists both tension and compression.

The layer 107 can be another radial gap layer similar to layer 111.

In some examples, the rigidizing devices described herein can have more than one rigidizing layer. For example, the rigidizing devices can include two, three, or four rigidizing layers. Referring to FIG. 3E, the layer 105 can be a second rigidizing layer 105. The second rigidizing layer 105 can have any of the characteristics described with respect to the first rigidizing layer 109. In some examples, the braid of second rigidizing layer 105 can be identical to the braid of first rigidizing layer 109. In other examples, the braid of second rigidizing layer 105 can be different than the braid of the first rigidizing layer 109. For example, the braid of the second rigidizing layer 105 can include fewer strands and have a larger braid angle α than the braid of the first rigidizing layer 109. Having fewer strands can help increase the flexibility of the rigidizing device 100 (relative to having a second strand with equivalent or greater number of strands), and a larger braid angle α can help constrict the diameter of the of the first rigidizing layer 109 (for instance, if the first rigidizing layer is compressed) while increasing/maintaining the flexibility of the rigidizing device 100. As another example, the braid of the second rigidizing layer 105 can include more strands and have a larger braid angle α than the braid of the first rigidizing layer 109. Having more strands can result in a relatively tough and smooth layer while having a larger braid angle α can help constrict the diameter of the first rigidizing layer 109.

The layer 103 can be another radial gap layer similar to layer 111. The gap layer 103 can have a thickness of 0.0002-0.04″, such as approximately 0.03″. A thickness within this range can ensure that the strands 133 of the rigidizing layer(s) can easily slip and/or bulge relative to one another to ensure flexibility during bending of the rigidizing device 100.

The outermost layer 101 can be configured to move radially inward when a vacuum is applied to pull down against the rigidizing layers 105, 109 and conform onto the surface(s) thereof. The outermost layer 101 can be soft and atraumatic and can be sealed at both ends to create a vacuum-tight chamber with layer 115. The outermost layer 101 can be plastic, a plastomer, or elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

In some examples, the outermost layer 101 can, for example, have tensile or hoop fibers 137 extending therethrough. The hoop fibers 137 can be made, for example, of aramids (e.g., Technora, nylon, Kevlar), Vectran, Dyneema, carbon fiber, fiber glass, boron, basalt, polyester, or other plastic. Further, the hoop fibers 137 can be positioned at 2-50, e.g., 20-40 hoops per inch. In some examples, the hoop fibers 137 can be laminated within an elastomeric sheath. The hoop fibers can advantageously deliver higher stiffness in one direction compared to another (e.g., can be very stiff in the hoop direction, but very compliant in the direction of the longitudinal axis of the rigidizing device). Additionally, the hoop fibers can advantageously provide low hoop stiffness until the fibers are placed under a tensile load, at which point the hoop fibers can suddenly exhibit high hoop stiffness.

In some examples, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, painting, or spraying the coating thereon.

The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

In some examples, the outermost layer 101 can be loose over the radially inward layers. For instance, the inside diameter of layer 101 (assuming it constitutes a tube) may have a diametrical gap of 0″-0.200″ with the next layer radially inwards (e.g., with a rigidizing layer). This may give the vacuum rigidized system more flexibility when not under vacuum while still preserving a high rigidization multiple. In other examples, the outermost layer 101 may be stretched some over the next layer radially inwards (e.g., the rigidizing layer). For instance, the zero-strain diameter of a tube constituting layer 101 may be from 0-0.200″ smaller in diameter than the next layer radially inwards and then stretched thereover. When not under vacuum, this system may have less flexibility than one wherein the outer layer 101 is looser. However, it may also have a smoother outer appearance and be less likely to tear during use.

In some examples, the outermost layer 101 can be loose over the radially inward layers. A small positive pressure may be applied underneath the layer 101 in order to gently expand layer 101 and allow the rigidizing device to bend more freely in the flexible configuration. In this example, the outermost layer 101 can be elastomeric and can maintain a compressive force over the braid, thereby imparting stiffness. Once positive pressure is supplied (enough to nominally expand the sheath off of the braid, for example, 2 psi), the outermost layer 101 is no longer is a contributor to stiffness, which can enhance baseline flexibility. Once rigidization is desired, positive pressure can be replaced by negative pressure (vacuum) to deliver stiffness.

A vacuum can be carried within rigidizing device 100 from minimal to full atmospheric vacuum (e.g., approximately 14.7 psi). In some examples, there can be a bleed valve, regulator, or pump control such that vacuum is bled down to any intermediate level to provide a variable stiffness capability. The vacuum pressure can advantageously be used to rigidize the rigidizing device structure by compressing the layer(s) of braided sleeve against neighboring layers. Braid is naturally flexible in bending (i.e., when bent normal to its longitudinal axis), and the lattice structure formed by the interlaced strands distort as the sleeve is bent in order for the braid to conform to the bent shape while resting on the inner layers. This results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some examples, the hoop fibers through or over the braid can carry tensile loads that help to prevent local buckling of the braid at high applied bending load.

The stiffness of the rigidizing device 100 can increase from 2-fold to over 50-fold, for instance 10-fold, 15-fold, or 20-fold, when transitioned from the flexible configuration to the rigid configuration. In one specific example, the stiffness of a rigidizing device similar to rigidizing device 100 was tested. The wall thickness of the test rigidizing device was 1.0 mm, the outer diameter was 17 mm, and a force was applied at the end of a 9.5 cm long cantilevered portion of the rigidizing device until the rigidizing device deflected 10 degrees. The forced required to do so when in flexible mode was only 30 grams while the forced required to do so in rigid (vacuum) mode was 350 grams.

In some examples of a vacuum rigidizing device 100, there can be only one rigidizing layer. In other examples of a vacuum rigidizing device 100, there can be two, three, or more rigidizing layers. In some examples, one or more of the radial gap layers or slip layers of rigidizing device 100 can be removed. In some examples, some or all of the slip layers of the rigidizing device 100 can be removed.

The rigidizing layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. In some of the apparatuses described herein, the variable stiffness layer comprises one or more filaments forming a plurality of filament regions that cross over each other and are free to move (e.g., slide) relative to one another in the flexible configuration, but may be increasingly constrained in the rigidizing state(s) when applying pressure (positive and/or negative pressure), which in some examples may drive a bladder layer against and/or into the variable stiffness layer. Other variable stiffness elements can be used in addition to or in place of the rigidizing layer. In some examples, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, longitudinal members or substantially longitudinal members.

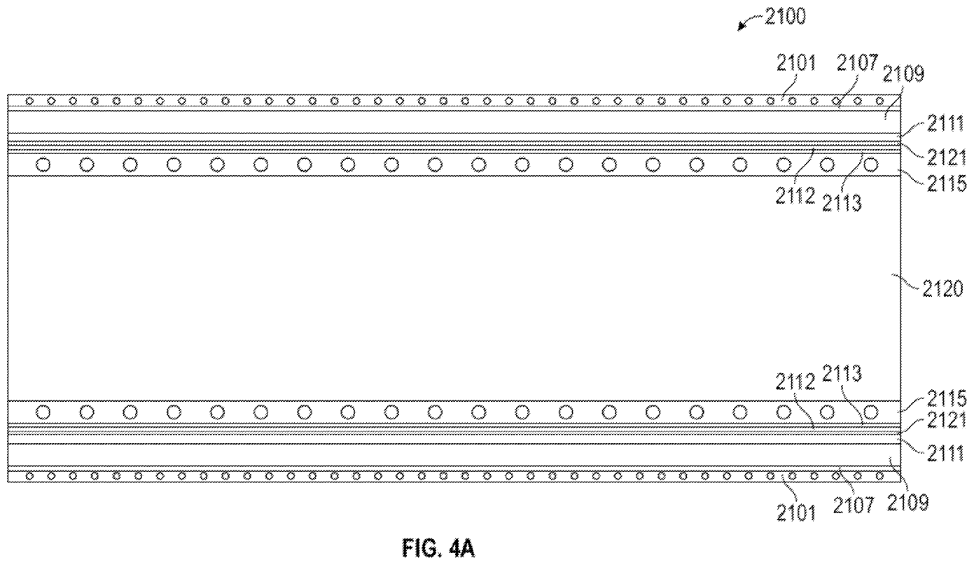

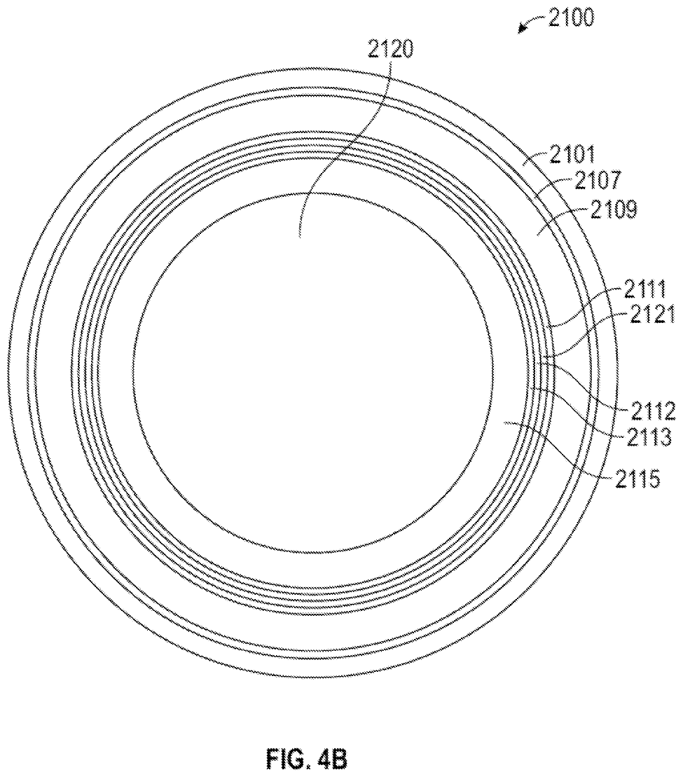

The rigidizable apparatuses described herein may also be rigidized by the application of positive pressure, rather than vacuum. For example, referring to FIGS. 4A-4B, the rigidizing apparatus (e.g., device or system) 2100 can be similar to rigidizing apparatus 100 described above, except that it can be configured to hold pressure (e.g., of greater than 1 atm) therein for rigidization rather than vacuum. A pressure-activated rigidizing device 2100 can also include a plurality of layers positioned around a lumen 2120 (e.g., for placement of an instrument or endoscope therethrough).

For example, FIGS. 4A-4B illustrate longitudinal and radial sections through an example of a pressure-activated rigidizable member of a rigidizing apparatus. The rigidizing device 2100 shown in FIGS. 4A and 4B can include an innermost layer 2115 (similar to innermost layer 115), a slip layer 2113 (similar to slip layer 113), a pressure gap 2112, a bladder layer 2121, a gap layer 2111 (similar to gap layer 111), a rigidizing layer 2109 (similar to rigidizing layer 109) or other variable stiffness layer as described herein, a gap layer 2107 (similar to layer 107), and an outermost containment layer 2101.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some examples, the pressure gap 2112 of the rigidizing device 2100 can be connected to a pre-filled pressure source, such as a pre-filled syringe or a pre-filled insufflator, thereby reducing the physician's required set-up time.

The bladder layer 2121 can be made, for example, of a low durometer elastomer (e.g., of shore 20 A to 70 A) or a thin plastic sheet. The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some examples, the bladder layer 2121 can be 0.0002-0.020″ thick, such as approximately 0.005″ thick. The bladder layer 2121 can be soft, high-friction, stretchy, and/or able to wrinkle easily. In some examples, the bladder layer 2121 is a polyolefin or a PET. The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the rigidizing layer 2109 against the outermost containment layer 2101 such that the relative motion of the braid strands is reduced.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other examples described herein. In some examples, the outermost containment layer 2101 can include a helical spring (e.g., made of circular or flat wire), and/or a tubular braid (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 include reinforcement elements therein, the rigidizing layer 2109 can be reasonably constrained from both shrinking diameter (under tensile loads) and growing in diameter (under compression loads).

By using pressure rather than vacuum to transition from the flexible state to the rigid state, the rigidity of the rigidizing device 2100 can be increased. For example, in some examples, the pressure supplied to the pressure gap 2112 can be between 1 and 40 atmospheres, such as between 2 and 40 atmospheres, such as between 4 and 20 atmospheres, such as between 5 and 10 atmospheres. In some examples, the pressure supplied is approximately 2 atm, approximately 4 atmospheres, approximately 5 atmospheres, approximately 10 atmospheres, approximately 20 atmospheres. In some examples, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

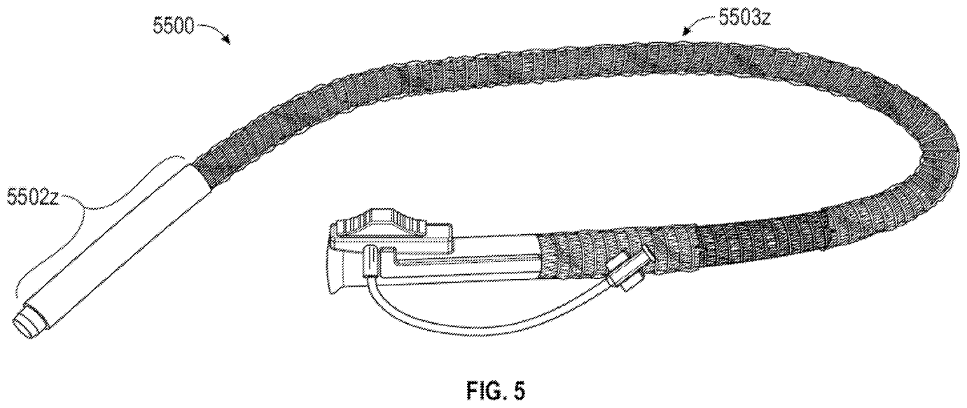

Any of the rigidizing devices described herein can have a distal end section or sections with a different design that the main elongate body of the rigidizing device. As shown in FIG. 5, for example, rigidizing device 5500 can have a main elongate body 5503z and a distal end section 5502z. Only the distal end section 5502z, only the main elongate body 5503z, or both the distal end section 5502z and the main elongate body 5503z can be rigidizing as described herein (e.g., by vacuum and/or pressure). In some examples, one section 5502z, 5503z is activated by pressure and the other section 5502z, 5503z is activated by vacuum. In other examples, both sections 5502z, 5503z are activated by pressure or vacuum, respectively.

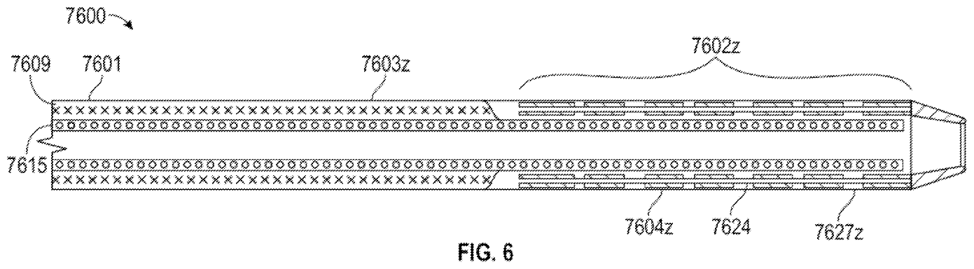

Referring to FIG. 6, in other examples, the distal end section 7602z can include a plurality of linkages 7604z that are actively controlled, such as via cables 7624, for steering of the rigidizing device 7600. The device 7600 is similar to device 5800 except that it includes cables 7624 configured to control movement of the device. While the passage of the cables 7624 through the rigidizing elongate body 7603z (i.e., with outer wall 7601, rigidizing layer 7609, and inner layer 7615) is not shown in FIG. 6, the cables 7624 can extend therethrough in any manner as described elsewhere herein. In some examples, one or more layers of the rigidizing elongate body 7603z can continue into the distal end section 7602z. For example, and as shown in FIG. 26, the inner layer 7615 can continue into the distal end section 7602z, e.g., can be located radially inwards of the linkages 7604z. Similarly, any of the additional layers from the rigidizing proximal section (e.g., the rigidizing layer 7609 or the outer layer 7601 may be continued into the distal section 7602z and/or be positioned radially inwards of the linkages 7604z). In other examples, none of the layers of the rigidizing elongate body 7603z continue into the distal section 7602z. The linkages 7604z (and any linkages described herein) can include a covering 7627z thereover. The covering 7627z can advantageously make the distal section 7602z atraumatic and/or smooth. The covering 7627z can be a film, such as expanded PTFE. Expanded PTFE can advantageously provide a smooth, low friction surface with low resistance to bending but high resistance to buckling.

FIG. 7A shows a section through one example of a proximal end region of a rigidizing aspiration sheath catheter 1130 showing the hemostatic valve region 1139 including a valve body 1131 and a pair of levers 1142, 1142′ for controlling opening and sealing the hemostasis valve of the rigidizing aspiration sheath catheter either closed or sealed over a device within the lumen of the rigidizing aspiration sheath catheter.

Referring now to FIGS. 7B-7D, various views of an embodiment of a hemostasis valve region of a rigidizing aspiration sheath catheter 1130 is shown. The perspective view of FIG. 7B, shows the valve body 1131. The valve body 1131 comprises a central bore 1172 at its proximal end. The central bore is in fluid communication with the lumen 1148 of the rigidizing aspiration sheath catheter 1130.

The device may also include a port for coupling to a positive or negative pressure (e.g., tube 1038 in FIG. 7B); this connector and tube may extend from the hemostatic seal 1132. The tube 1139 is connected to the bladder layer of the rigidizing catheter 1130. As shown in FIG. 7B, the rigidizing aspiration sheath catheter may also include a flush line 1034, e.g., as a tube extending from hemostatic seal 1132. The tube 1034 connects to the flush port and allows flushing of the working lumen of the rigidizing catheter.

In general, any of the apparatuses described herein may include a flush port. The flush port may be particularly advantageous, as it may allow for flushing of the catheter, and may also be used to inject contrast and for continuous hemodynamic measurements.

In FIG. 7B, the levers 1142, 1142′ extend distally from a proximal portion of the valve body. The levers 1142, 1142′ can be biased outwards which corresponds to the valve 1170 within the seal being closed, as shown in the end view of FIG. 7C. Springs (e.g., torsion springs, leaf springs, compression springs tension springs, etc.) can be used to bias the levers outwards. Actuating the levers 1142, 1142′ by depressing them inwards, towards the central bore, opens the valve 70 and allows insertion of devices (e.g., aspiration catheter) into the valve central bore and rigidization catheter or removal of devices (e.g., aspiration catheter) from the rigidization catheter and the valve central bore. This lever-actuated design allows a user to operate the valve using a single hand. The valve can comprise an annular seal (e.g., annual elastomeric seal) configured to seal off the central bore of the valve or seal around a device positioned within the central bore of the valve.

The amount of sealing achieved by the hemostasis valve 1170 can advantageously be dynamically adjusted during use (e.g., during a procedure with an aspiration catheter) by adjusting the position of the levers 1142, 1142′. For example, the user may feel the tactile feedback when operating the actuator, indicating the compression level of the soft annular elastomeric seal, and may adjust the seal, e.g., to reduce or increase drag on a medical device within the hemostatic valve as desired.

Devices to be inserted within the valve central bore 1172 and rigidizing catheter can comprise a proximal connector with a mating mechanism configured to attach to a corresponding mating mechanism 1164 at the proximal end of the valve 1132 to stabilize the device relative to the valve. In some embodiments, the mating mechanism comprises a bayonet feature. Other mating mechanisms (e.g., threaded connection) are also contemplated.

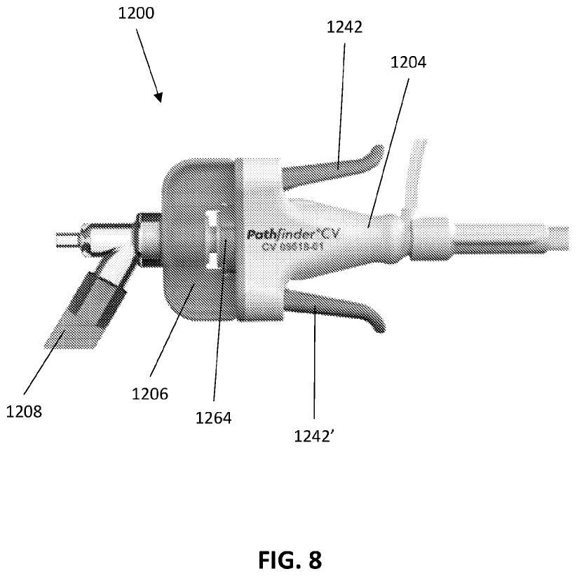

FIG. 8 illustrates an embodiment of a handle region 1200 of a rigidizing aspiration sheath catheter. In this example the aspiration catheter handle 1200 can be positioned at a proximal end of a rigidizing aspiration sheath catheter (not shown). In some embodiments, the connection between the rigidizing aspiration sheath catheter and the aspiration catheter handle can be a luer style connection. The aspiration catheter handle region can include a central body 1204 comprising a valve and central bore (not shown), similar the hemostatic seal, described above. The aspiration catheter handle may include actuators (e.g., levers 1242, 1242′), similar to levers 1142, 1142′, described above). The levers 1204 extend distally from a proximal portion of the valve body. The levers 1204 can be biased outwards, which corresponds to the valve within the handle being closed. Springs (e.g., torsion springs, leaf springs, compression springs tension springs, etc.) can be positioned and configured to bias the levers outwards. Actuating the levers 1204 by depressing them inwards and allows connection of a connector 1206 of a vacuum line 1208. This lever-actuated design allows a user to operate the valve using a single hand.

The amount of sealing achieved by the hemostasis valve region can advantageously be adjusted during use (e.g., during a procedure with an aspiration catheter) by adjusting the position of the levers 1242, 1242′. For example, the user may feel the tactile feedback when operating the actuator, indicating the compression level of the soft annular elastomeric seal, and may adjust the seal, e.g., to reduce or increase drag on a medical device within the hemostatic valve as desired.

The vacuum line connector 1206 can comprise a mating mechanism configured to attach to a corresponding mating mechanism 1264 at the proximal end of the valve to stabilize the device relative to the valve. In some embodiments, the mating mechanism comprises a bayonet feature. Other mating mechanisms (e.g., threaded connection) are also contemplated.

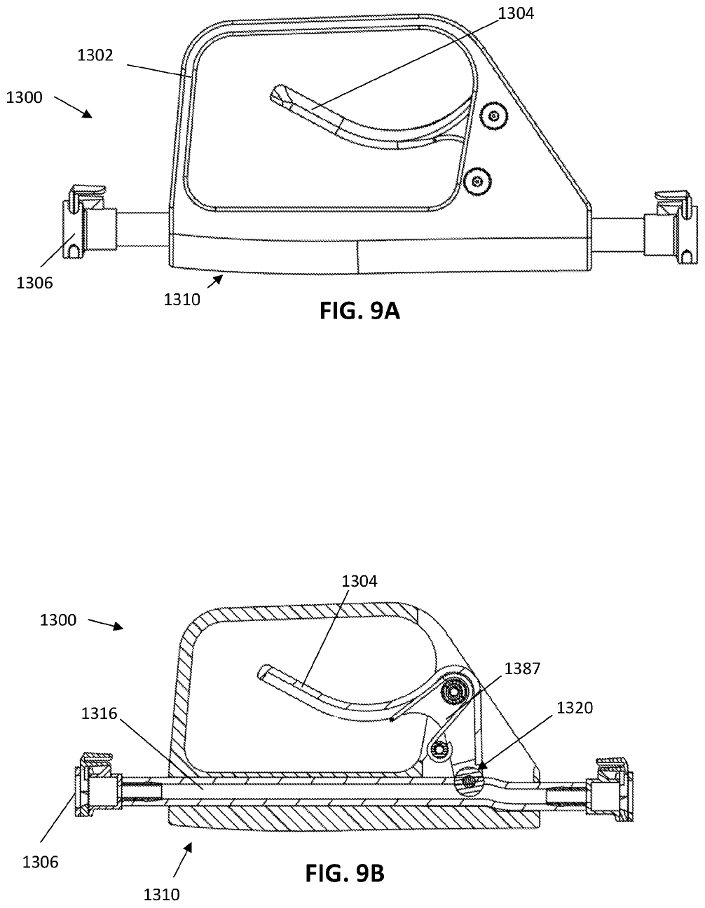

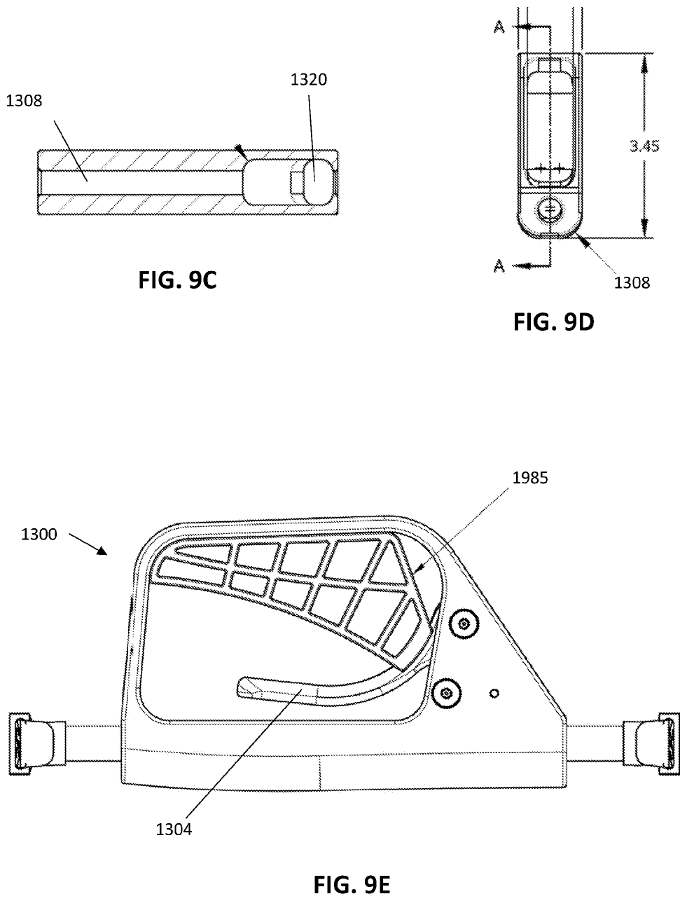

Also described herein are hand-triggered vacuum activation valves. FIGS. 9A-E illustrate example views of an embodiment of a vacuum activation valve 1300. The hand-triggered vacuum activation valve 1300 includes a handle region 1302 and actuating lever 1304. FIG. 9A shows a side view of the vacuum activation valve 1300. FIG. 9B shows a side sectional view. FIG. 9C shows a sectional view taken through the base region of the handle shown in FIGS. 9A and 9B. A front view is shown in FIG. 9D.

The hand-triggered valve 1300 includes a lower portion 1310 through which the vacuum line 1316 extends. A groove or channel 1308 configured to receive that vacuum line is shown in the section view of FIG. 9C. A clamp 1320 is positioned over the channel 1316. The clamp 1310 is configured to clamp down on the vacuum line when the lever 1304 is actuated. In some examples, the clamp 1320 is configured to unclamp the vacuum line when the lever 1304 is actuated. In the un-actuated state the clamp may be configured to pinch down on (and close off) the vacuum line.

Positioned above the lower portion is a handle 1302 comprising a loop extending distally from a proximal portion of the handle. The loop can be sized to be able to be gripped within the palm of a user. The lever 1304 extends proximally from a proximal portion of the hand-operated valve 1300. The lever 1304 is sized such that it can be actuated by one or more fingers of a user while the handle 1302 is being gripped within the palm of the user. A distance between the handle 1302 and the 1304 is selected to enable the lever 1304 to be actuated when the user is gripping the handle 1302. In some embodiments, the distance is about 0.3-1.5 inches. The loop (as shown in the side view) may comprise a number of shapes (polygonal, rectangular, ovular, etc.).

In FIGS. 9A-9D the lever 1304 is shown in an unactuated state, so that the clamp pinches the channel 1316 shut. In some examples the lever and/or the clamp may be biased in the closed state by a bias 1387 (shown as a spring bias). When the lever is actuated, e.g., by pushing it down, the clamp 1320 may be pulled open, away from the channel 1316, allowing suction to pass.

In some cases it may be helpful to store a vacuum activation valve 1300 with the control (e.g., lever 1304) in a closed/actuated position. This may prevent deformation of the valve (e.g., clamp, crip, etc.) mechanism and/or the channel 1316. For example, FIG. 9E shows an example of a vacuum activation valve 1300 in which a retainer 1395 is coupled thereto, to hold the lever 1304 in a closed position, maintaining the channel within the vacuum activation valve in an open position. The retainer may be removed before use, e.g., once connected to the fluid line.

The valve can have any appropriate length. In some examples the valve has a length of about 4-6 in (or about 3-5, 5-7, 5-6 in., etc.). The handle may be any appropriate length. For example, the length of the handle can be about 3-5 in. (or about 2-4 in., 4-6 in. 4-5 in., etc.). The length of the lever can be, e.g., about 3-5 in. (or about 2-4 in., 4-6 in. 4-5 in., etc.). The lever can be curved as shown in FIG. 9A. In other embodiments, it is straight or angular.

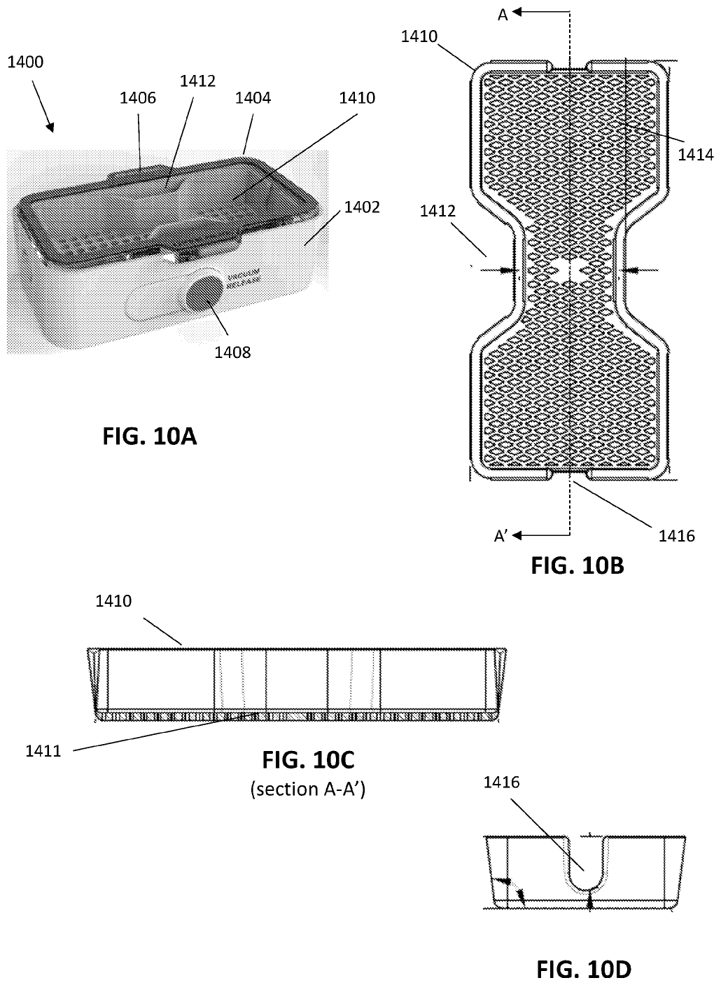

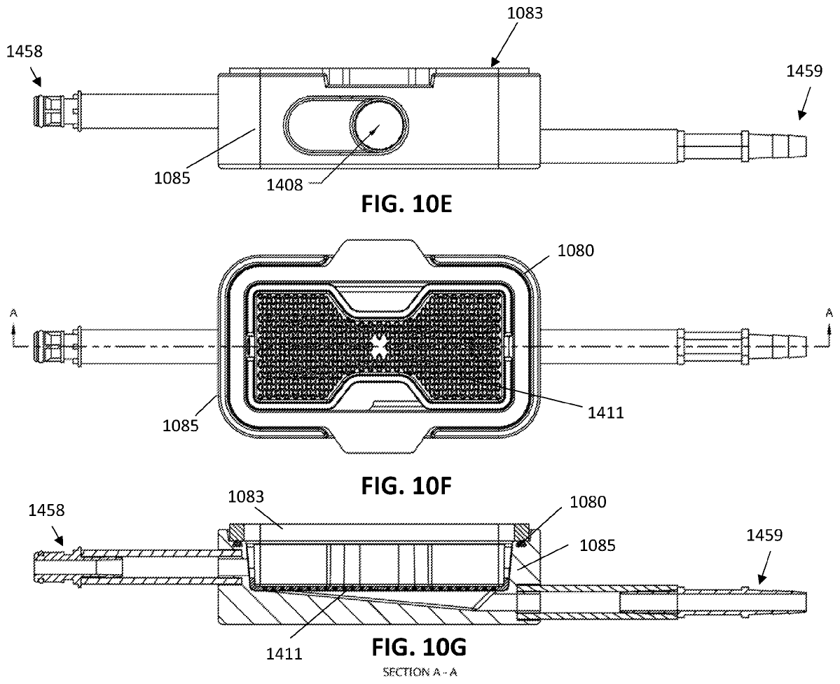

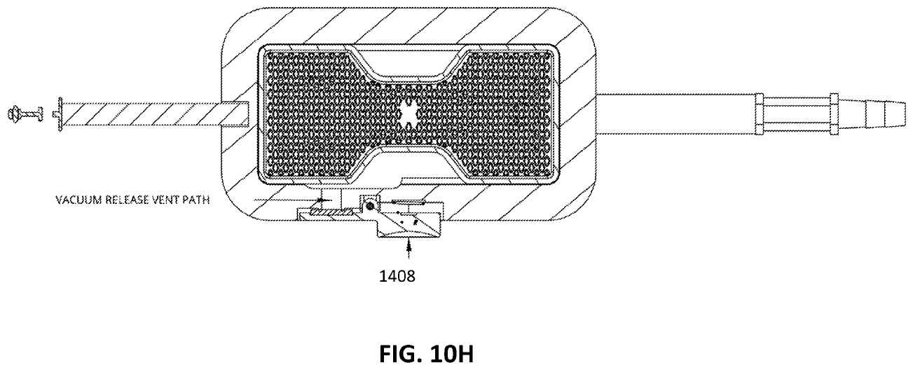

Also described herein are clot capture chambers. FIGS. 10A-D show views of an example of an embodiment of a clot capture component (e.g., like that shown in FIG. 1B). FIG. 10A is a perspective view of a clot capture component. The clot capture component 1400 comprises a container 1402 with a lid 1404. The lid 1404 can be completely removed from the container 1402. In some embodiments, the lid is connected to the container by a hinge. The lid 1404 can comprise one or more tabs 1406 extending or projecting from an edge of the lid to aid in lifting and/or removal of the lid. In some embodiments, the lid is transparent, which can advantageously allow for visualization of aspirated material therethrough.

When under negative pressure from an attached vacuum source, the lid may be sealed to the container. To enable removal of the lid, a user may perform a vent to atmosphere to release the negative of pressure within the container. In some embodiments, this venting is controlled by a button 1408 positioned on the container.

The container 1402 can have a generally rectangular prism shape. Other shapes are also possible (e.g., square prism, elliptic cylinder, etc.). In some embodiments, the container 1404 comprises a length of about 3-5 in. (or about 2.5-5.5 in., 3.5-4.5 in., about 4 in., etc.). The container can have a width of about 1-3 in. (or about 0.75-3.25 in., 1.5-2.5 in., about 2 in., etc.). The container can have a depth of about 0.3-1.1 in. (or about-. 4-1.0 in., 0.5-0.9 in., 0.6-0.7 in., etc.).

Positioned within the container is a tray 1410. FIG. 10B shows a top view of the tray 1410. FIG. 10C shows a section view taking along line A-A of FIG. 10B. FIG. 10D shows a front view of the tray 1410. In some embodiments, the tray 1410 is removable from the container 1402. The tray 1410 is sized to conform to an inner perimeter of the container 1402. The tray 1410 may have one or more regions 1412 where it is spaced away from an inner perimeter of the container 1402 to enable easy removal of the tray 1410 from the container.

The tray 1410 includes one or more intermediate surface(s) 1411 having apertures 1414 to allow for draining of blood and other fluids. In some embodiments, the intermediate surface comprises a screen or mesh like configuration. The apertures may be any sized to permit fluid (e.g., blood) to flow through, but retain clot material. The apertures (e.g., mesh) may be any appropriate size. For example, the apertures may be between about 0.037 mm and about 6 mm (e.g., between about 0.05 mm and about 5 mm, between about 0.1 mm and about 4 mm, between about 0.2 mm and 3 mm, between about 0.3 mm and 2.5 mm, between about 0.5 mm and about 4 mm, between about 0.75 mm and about 4 mm, etc.). In some examples the intermediate surface may include multiple layers of mesh having different mesh sizes in order to progressively filter out smaller and smaller clot sizes without clogging the chamber. The different layers may be separated by a gap (e.g., of between about 0.5 mm to 2 cm). These mesh regions may be removable to allow analysis and/or capture of the different size clots and/or cleaning (including sterilization).

The tray 1410 may include channels 1416 along two opposing sides to allow access to a vacuum lumen. The channels 1416 may be U-shaped. This shape may allow the channels to partially surround a vacuum aperture on a side of the container.

The tray can have a length of about 3-5 in. (or about 2.5-5.5 in., 3.5-4.5 in., about 4 in., etc.). The tray can have a width of about 1-3 in. (or about 0.75-3.25 in., 1.5-2.5 in., about 2 in., etc.). The tray can have a depth of about 0.3-1.1 in. (or about-. 4-1.0 in., 0.5-0.9 in., 0.6-0.7 in., etc.).

In some embodiments, the tray comprises a polymer, such as acrylonitrile butadiene styrene (ABS).

FIGS. 10E-10H show side, top, side section and top section views, respectively of an example of a clot capture chamber as described herein. In FIG. 10E a vacuum release valve 1408 is positioned on the side of the chamber the device may be connected in-line as part of the vacuum line, so that fluid is drawn in from the top inlet line 1458, so that the fluid (e.g., blood with clot material) enters the upper region of the chamber and into the tray; the blood may then drain to the bottom of the chamber and out of the outlet 1459, as shown in the sectional view of FIG. 10G. In any of these clot capture chambers the inlet may be positioned at a top region, and the outlet may be positioned at the bottom region, allowing gravity to assist in passing the blood through the porous intermediate layer(s). Any of these apparatuses may include a seal (e.g., gasket) between the main body and the lid, as shown in section view 1080.

An exemplary method of using the aspiration system described herein comprises navigating through the vasculature to or near a desired clot treatment site using a guidewire (e.g., 0.014″ or a 0.035″ wire). A rigidizing catheter can then be advanced, in a flexible state, over the guidewire to or near the clot treatment site. Once at or near the site, the rigidizing catheter may be transitioned to the rigid state. An obturator can be used during advancement of the rigidizing catheter. At this point, the guidewire may be removed, if so desired. Removing the guidewire can advantageously free up space within the lumen of the rigidizing catheter.

Once the rigidizing catheter is in the rigid space, it creates a stable conduit or platform from which treatment can be initiated. An aspiration catheter can be advanced through the rigidizing catheter. The aspiration catheter can have a proximal connector configured to lock to the hemostasis valve of the rigidizing catheter. The aspiration catheter can have a bigger inner lumen than traditional aspiration catheters because the stable platform created by the rigidizing catheter reduces the need for the aspiration catheter to perform accurate navigation and allows it to have a larger central bore. As mentioned here, the rigidizing catheters described herein may provide sufficient support (e.g., mechanical integrity) to one or more additional devices or components to allow these additional components (guidewires, aspiration catheter, stent, heart valve, tools, etc.) to be more flexible than other components. The stiffness necessary for the control and operation of a standalone component may become redundant when used within the rigidizing catheters described herein, which may provide the mechanical support that is otherwise required in the standalone component.

In some embodiments, the aspiration catheter can be switched out for a different aspiration catheter (e.g., comprising a different size). The hemostasis valve can be actuated to release the first aspiration catheter and insert the second aspiration, allowing for a quick exchange of the aspiration catheters. The aspiration catheter handle can be actuated to release the first aspiration catheter from the vacuum line and a second aspiration catheter can be connected to the vacuum line.

In some embodiments, the aspiration catheter may be removed to allow for flushing of the aspiration line. The rigidization catheter maintaining the stable pathway to the treatment site allows for this flushing to occur without losing the catheter position.

In some embodiments, the method further comprises visualizing aspirated material through the transparent lid of the clot capture container. In some embodiments, the container may be vented, and the lid removed. The tray may be replaced with another clean tray, for example, if the container is getting full. Traditional aspiration systems do not allow for a large enough aspiration lumen size to aspirate enough material to require this replaceable tray feature. A single tray can be used throughout the procedure, or multiple trays can be used—for example, one tray for each suction attempt or for each clot removed.

In some embodiments, aspiration can be performed directly through the rigidizing catheter.

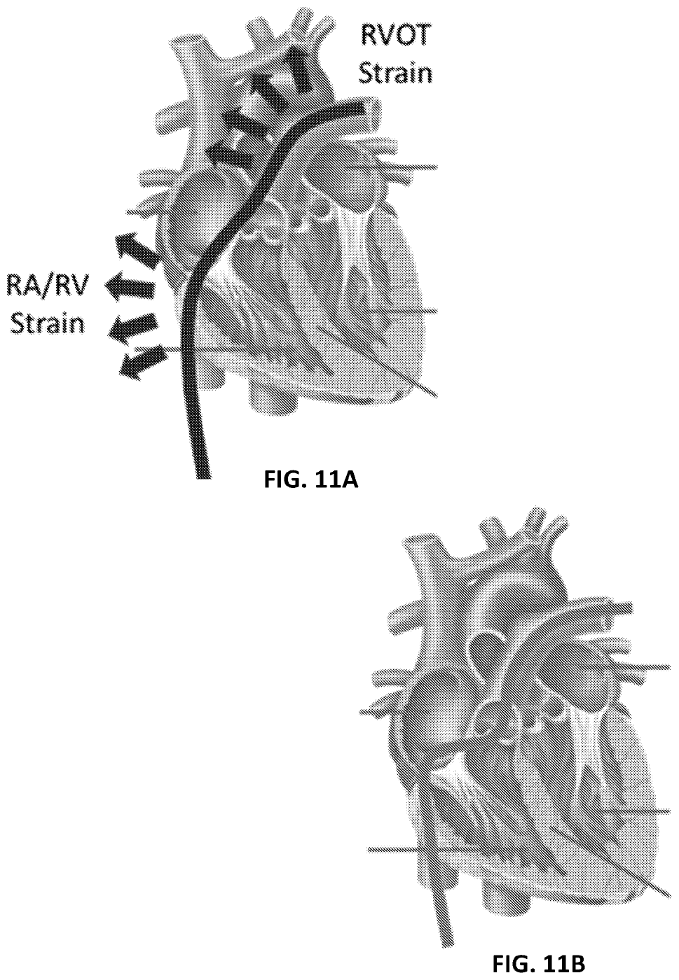

FIGS. 11A and 11B show a traditional catheter navigated to the pulmonary artery (FIG. 11A) and the rigidizing system described herein navigated to the pulmonary artery (FIG. 11B). As shown in FIGS. 11A and 11B, the rigidizing system's ability to establish the pathway in a flexible state leads to reduced strain and reduced potential for hemodynamic compromise and injury.

In general, the removal of clot material may result in the loss of large blood volumes. Blood loss can be associated with the duration of the aspiration and the proximity of the aspiration catheter tip to the clot. However, the methods and apparatuses described herein may be configured to minimize blood loss. For example, the use of rigidizing apparatuses as described herein may allow apparatuses to be positioned proximate to the clot, which may minimize the blood volume required to move the clot. The stabilization achieved with the rigidizing catheters described herein may improve access to the clot and leads to better engagement between the tip of the aspiration catheter and the clot, resulting in lower blood loss by virtue of early and improved clot engagement. In addition, the methods and apparatuses described herein may be used without the use of guidewires. In particular, the apparatuses described herein may be configured to accommodate a cardiotomy reservoir (e.g., a suction canister/blood filter), which may be integrated within a thrombectomy circuit as described herein.

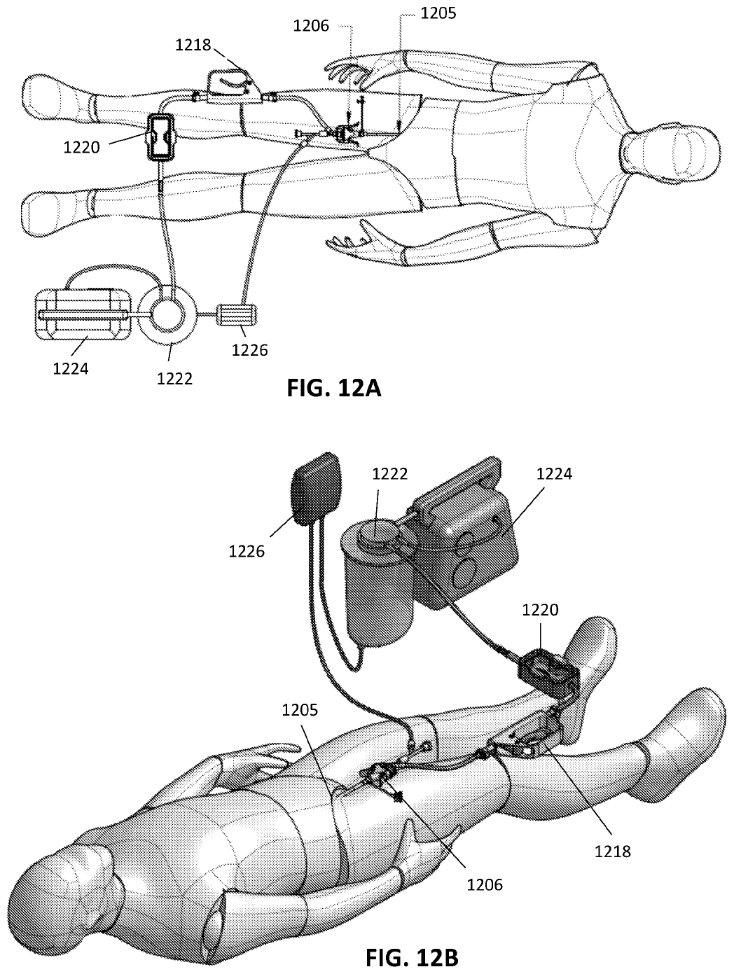

FIGS. 12A-12B, 13A-13B, 14A-14B, 15A-15B and 16A-16B illustrate examples of systems including a rigidizing aspiration sheath catheter configured with a blood return circuit. In general, these apparatuses may be configured for use with a patient to remove, aspirate material, and in particular clot material, from the vascular system of a patient. In any of these examples the apparatus may include a return circuit for returning and/or replacing blood removed during the procedure. For example, FIG. 12A illustrate a system as described herein including any of the rigidizing aspiration catheters 1206 described herein. The aspiration catheter may be coupled to a hand-operated valve (extraction handle 1218) that connects the aspiration tube in-line with the clot capture container 1220, which is in turn connected in-line with a suction canister including one or more blood filters 1222. The suction cannister may be held under vacuum to a negative pressure set by a vacuum pump 1224.

The suction cannister may filter and/or treat the blood so that it may be reintroduced into the body. For example the suction canister 1222 may include filtration to remove clot material and/or may be treated with one or more agents to reduce or prevent infection and/or to reduce and prevent clotting (e.g., anticoagulants). As mentioned above, the optional clot capture 1220 device may also filter clot material before it reaches the suction canister 1222. In general the filtered and/or treated blood may be reintroduced back into the body either directly or indirectly, e.g., by first passing to a blood bag 1226. Supplemental blood may be provided.

In general, the sterility of the operating sterile field may be maintained by keeping all of these elements (e.g., the blood bag, suction cannister, clot capture device, handle, etc.) within the sterile field. Any or all of these components may be single-use and/or reusable (and sterilizable).

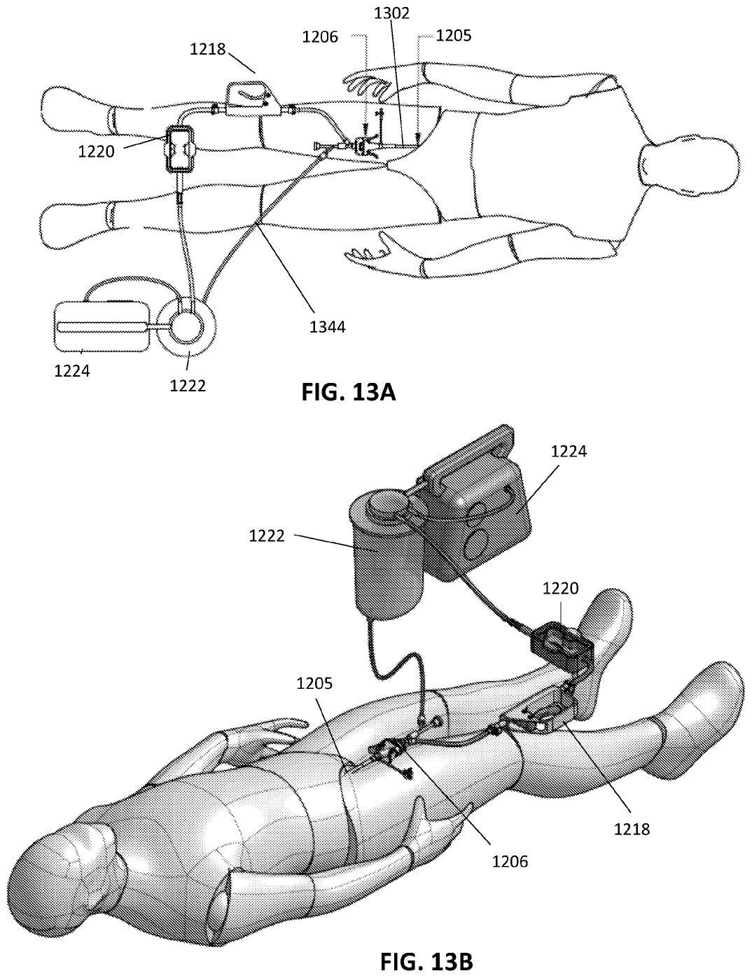

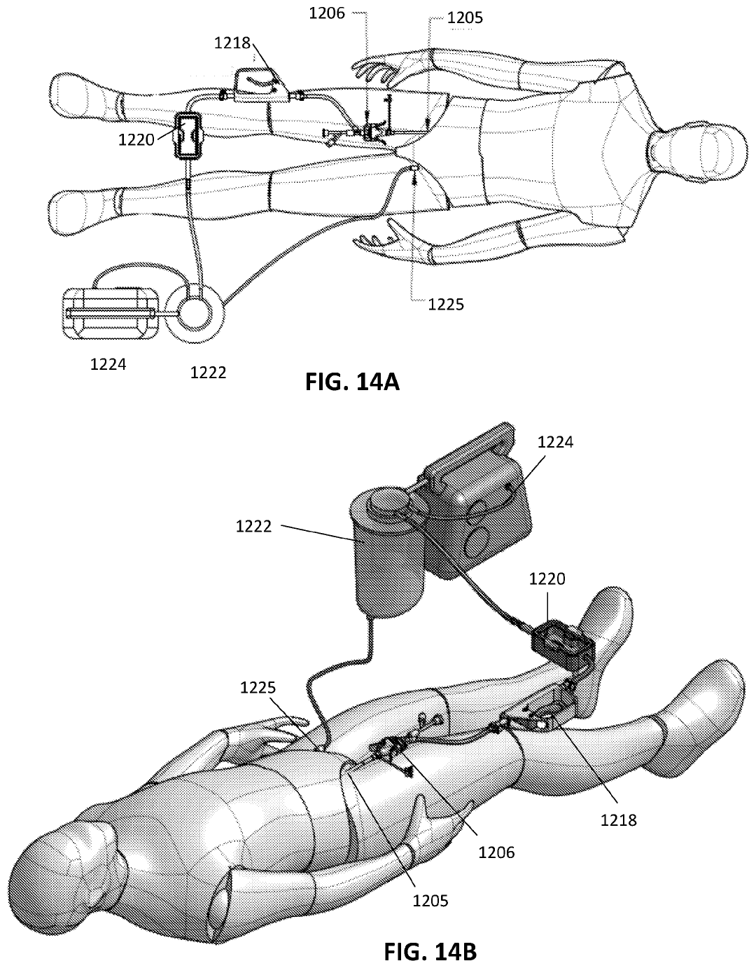

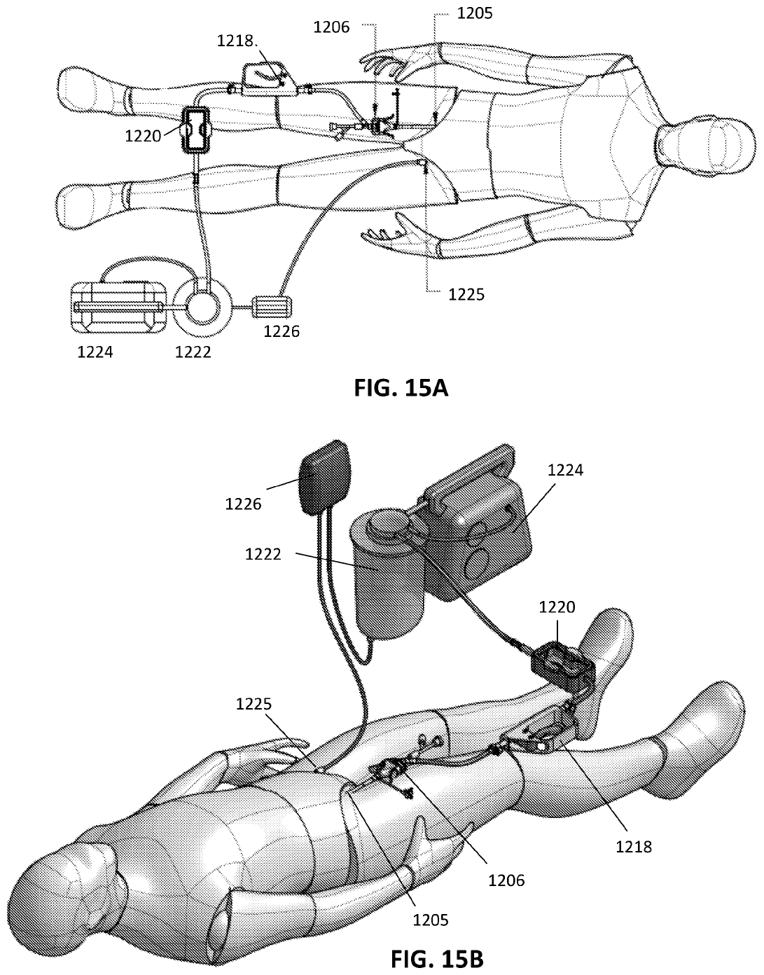

The rigidizing aspiration catheter may be inserted into an access site 1205 on the patient's body, such as the femoral artery. Note that any appropriate access region may be used (e.g., radial, ulnar, axillary, brachial, dorsalis pedis, posterior tibial). In FIGS. 12A-12B and 13A-13B the system is configured so that the material, including blood, is removed and returned via the same access site 1205. Alternatively, in some examples the return site may be a separate access site, such as illustrated in FIGS. 14A-14B and 15A-15B. In these examples the blood return site 1225 may be on the contralateral side (e.g., the contralateral femoral artery).

For example, FIGS. 13A-13B illustrate a configuration of an apparatus including a rigidizing aspiration sheath catheter 1302, which includes a hemostasis valve at the proximal end coupled to a suction connector 1206. The suction connector fluidically connects to the control valve (e.g., configured as an actuation handle 1218), in-line with a clot capture chamber 1220 and a blood capture/filter chamber 1222 that is connected to a source of vacuum 1224, similar to FIGS. 12A-12B. In this example, the rigidizing aspiration sheath catheter may be inserted through the access site 1205 and navigated through the body (e.g., over a guidewire) including as shown in FIGS. 11A-11B, across the heart, to a target region to remove clot. Once in position, the rigidizing aspiration sheath catheter may be rigidized, e.g., by applying positive and/or negative pressure, and coupled to the suction line using the connector 1206. Aspiration may be applied, e.g., by actuating the control valve 1218, with the device maintained in the rigid configuration (e.g., by maintaining the positive and/or negative pressure).

In some examples, after aspirating one or more times, by activating and releasing the extraction handle 1218, a state of vacuum may be re-established between extraction handle and the blood capture chamber 1222 (e.g., reservoir), including the portion of the blood return line up to the patient; a check valve may be included on the blood return line. Blood may be held by the blood capture region. In any of these examples, an optional blood return circuit may be included, as shown. In this example, the blood return circuit includes a return tubing line 1344.

In any of these methods, the rigidizing aspiration sheath catheter may be used to support a second catheter, an aspiration catheter, which may be inserted through the rigidizing aspiration sheath catheter, as described above. The rigidizing aspiration sheath catheter may be uncoupled from the suction line, the aspiration catheter inserted through the rigidizing aspiration sheath catheter (e.g., using the posterior hemostasis valve of the rigidizing aspiration sheath catheter) and the aspiration catheter coupled to the suction line, all while the rigidizing aspiration sheath catheter remains in the rigid configuration. Suction may then be applied through the aspiration catheter, while the rigidizing aspiration sheath catheter remains rigid, to support the aspiration catheter.

When it is time to reinfuse blood to the patient the vacuum may be removed (e.g., bringing the system to atmospheric pressure) which may be done by pressing a release control (e.g., button) on the clot capture chamber 1220, which may leak air into the system in a controlled manner. Air and any remaining blood may be in the clot capture chamber 1220 may be driven to the blood capture chamber/filter 1222. In some cases the lid of the clot capture chamber may be taken off to maintain this state. At this point, a user could use a syringe at the check valve to pump/reinfuse blood to the patient, or a more automated blood return may be used.

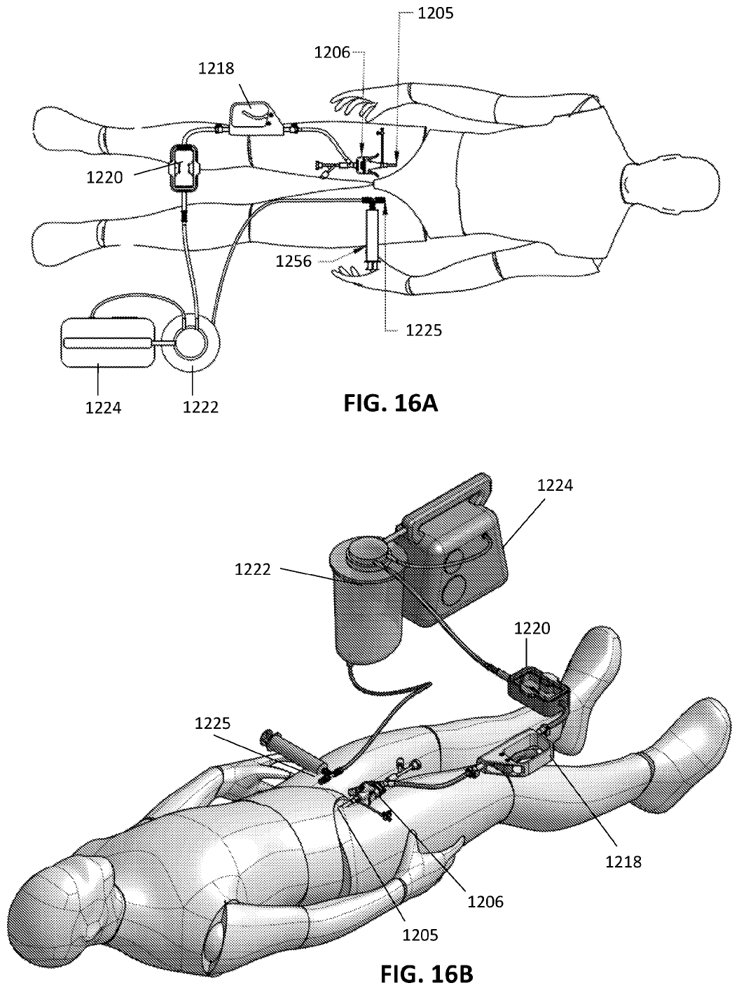

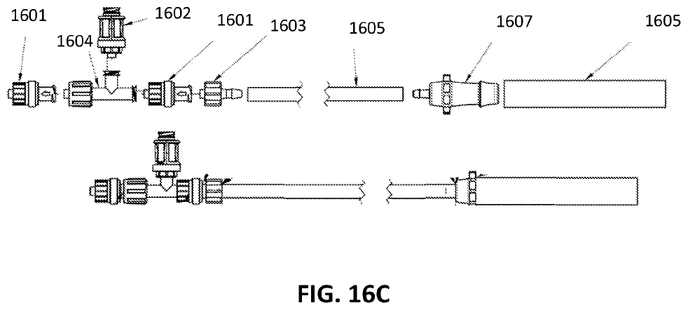

As mentioned, in some examples the blood return circuit may be direct, and may couple from the suction canister/blood filter directly back into the patient's body, as shown in FIGS. 13A-13B and 14A-14B. For example, blood may exit the patient from the catheter and may be returned to patient through either the catheter (e.g., the rigidizing aspiration sheath catheter or aspiration catheter) or alternate means (not shown). FIGS. 16A-16B illustrate an alternative site 1225 for re-introduction of blood (e.g., on the opposite leg) using a syringe 1256, as mentioned above. FIG. 16C shows an example of a return assembly 1600 including a check valve 1601, a swabble luer fitting 1602, a male luer lock 1603, tubing 1604, 1605, and a T-connector 1506 and a reducer 1607. This blood return tubing assembly may generally include multiple one-way valves arranged so that fluid may be injected into the body while minimizing blood loss.

As mentioned, the order of the components of the blood circuit may be different.

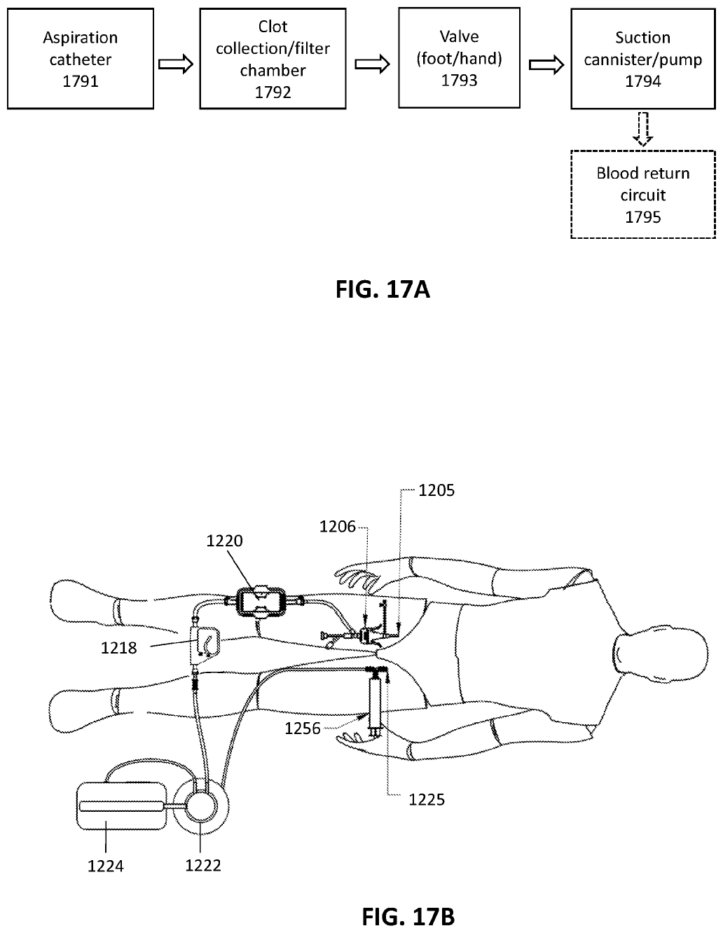

FIGS. 17A-17B show an example of a system in which the clot capture chamber 1220 positioned proximal to the suction valve 1218. For example, in FIG. 17A, the rigidizing aspiration sheath catheter and/or aspiration catheter 1791 may be proximal to the clot collection chamber 1792 which is proximal to the suction valve 1793 (e.g., shown as a hand valve 1218 in FIG. 17B) and the blood collection chamber (e.g., suction cannister) and pump 1794.

As mentioned above, the methods and apparatuses described herein may also improve the steering (e.g., guidance) and operation of suction catheters applied through the rigidizing aspiration sheath catheters described herein. For example, the use of dynamic rigidization by the rigidizing aspiration sheath catheter provide a sable pathway through the vasculature of the body, and may be used without a guidewire, or with a highly flexible guidewire, where other systems may require the use of guidewire and/or more rigid guidewires. This is because the rigidizing aspiration sheath catheter may be frozen and/or locked (or in some cases just partially stiffened) into a shape or pathway that remains relatively fixed. In the stiffer configuration the apparatus may provide a reference platform against which the catheter inserted through the rigidizing aspiration sheath catheter (e.g., an aspiration catheter) may be driven. For example, when steering an aspiration catheter, one or more pull wires may be used to change the orientation of the catheter, including to angulate a tip; for the angulation to occur at the distal end of the catheter, the proximal end of the catheter must remain relatively stiff enough to overcome total tension of pull wire. Thus, this may limit the overall flexibility of the catheters used. In contrast the rigidizing aspiration sheath catheters described herein may provide the necessary support, when rigidized, to allow for the sufficient mechanical advantage. Thus, a highly flexible aspiration catheter may be used with a rigidizing aspiration sheath catheter that may support the proximal end of the more flexible aspiration catheter.

A rigidizing aspiration sheath catheter may also act as a stable platform to allow mechanical support for other devices, such as (but not limited to) graspers, imaging system, probes, guidewires, or the like. The distal end of the rigidizing aspiration sheath catheter, when rigidized, may act as a control point for such instruments.

Any of the apparatuses described herein may be used in a fully rigidized or partially rigidized configuration. For example, the rigidity of the rigidizing aspiration sheath catheters described herein may be adjusted by increasing or decreasing the applied pressure. Typically the higher the pressure (higher positive pressure in some examples), the more rigid the rigidizing aspiration sheath catheter can become. This may allow the rigidizing aspiration sheath catheter to be partially stiffened. Further, the fac that the rigidizing aspiration sheath catheter may be restored to a highly flexible configuration may allow these devices to be inserted and removed without risking damaging the subject's anatomy. In practice, excessively stiff systems typically lead to vascular and hemodynamic complications, and frequently prevent reliable access to anatomies at the end of tortuous pathways. The rigidizing aspiration sheath catheters described herein may avoid such concerns by providing an anatomically conformable pathway that does not rely on anatomical support and/or accommodation while greatly extending the reach and stability to anatomies that have been difficult to access. This may allow users (e.g., doctors, technicians, etc.) to better plan their maneuvers, without having to anticipate where the anatomy will be based on catheter movement), and may provide an added level of safety by reducing the urgency that may result from the uncertainty caused by a moving target.

The rigidizing aspiration sheath catheters may also protect the adjacent anatomy. Once the rigidizing aspiration sheath catheter device is located and rigidized within the body, subsequent instruments introduced through the system are less likely to straighten the access pathway leading to unintended forces on the adjacent anatomy. In its flexible state, the rigidizing aspiration sheath catheter typically follows the patient anatomy. In its rigid state, the rigidizing aspiration sheath catheter reduces the tendency for inadvertent changes in the pathway. For example, a conventional introducing catheter has a defined flexibility. That sheath may be placed through the heart and the heart may accommodate it. However, when additional instruments are passed through the traditional sheath, the stiffness increases and acts to stiffen the overall system. The resulting straightening can deleteriously affect adjacent anatomical structures, potentially increasing tension on the right ventricle or exacerbating tricuspid valve regurgitation. In contrast, once the rigidizing aspiration sheath catheter is placed and rigidized within the anatomy, the ability of any additional instruments to straighten the access pathway is greatly reduced or eliminated. Consequently, the patient remains hemodynamically stable after insertion and rigidizing of the rigidizing aspiration sheath catheter, because there will not be further changes in the anatomical pathway while the rigidizing aspiration sheath catheter is stabilized, and the anatomy is isolated.

In addition, the rigidizing aspiration sheath catheters described herein may allow multiple exchanges with inserted tools without losing position. This allows flexibility and opportunity by freeing the procedure from the requirements for a guidewire, and allows the use of highly “soft” or flexible guidewires (e.g., guidewires having a stiffness of 10 GPa or less, e.g., 9.5 GPa or less, 9 GPa or less, 8.5 GPa or less, 8 GPa or less, 7.5 GPa or less, 7 GPa or less, 6.5 GPa or less, 6 GPa or less, 5.5 GPa or less, 5 GPa or less, 4.5 GPa or less, 4 GPa or less, 4 GPa or less, 3 GPa or less, 2 GPa or less, 1 GPa or less, etc., where stiffness corresponds to the flexural modulus in gigapascals, GPa) than about than may otherwise be possible with other catheters and catheter sheaths. This may improve the safety of the procedure by reducing the potential for perforation or laceration associated with stiff and extra stiff guidewires. Because the rigidizing aspiration sheath catheter maintains is position, a role normally relegated to the guidewire, guidewires can be easily exchanged and replaced without loss of position.

The rigidizing aspiration sheath catheters described herein may be dynamically rigidized to allow rapid positioning and repositioning. Thus, complex catheter procedures are possible, and the rigidizing aspiration sheath catheter may reduce or eliminate ancillary motion due to a stack-up of various flexible systems by stabilizing the entire system in the rigid configuration. This may result in reduced overhead and may simplify endovascular procedures.

### Relative Stiffness

In general, the rigidizing aspiration sheath catheters described herein may be transitioned from a highly flexible configuration to a more rigid configuration. In the rigid state, the rigidizing aspiration sheath catheter may have a higher stiffness than other aspiration catheters, which may allow for significantly more robust operation, providing a functional advantage of the system over other devices. Localized flexural stiffness measurements on traditional aspiration catheters (e.g., stiffness at localized points along the length of the catheter) as compared to the rigidizing aspiration sheath catheter in a rigid configuration have shown that the EI (e.g., Young's modulus x bending moment of inertia), or measurement in units of lbf-inch² show a significant difference. For example, table 1, below shows measured EI values of a 20 F aspiration catheter and a 26F rigidizing aspiration sheath catheter, compared with a commercially available suction catheter (“traditional” catheter, shown as an INARI™ 20 F catheter). The units in table 1 are stiffness EI (lbf-inch²):

TABLE 1

| | | rigidizing | | | |
|---|---|---|---|---|---|
| | | aspiration sheath | | | |
| | | catheter | | | Tradi- |
| | | Flexible | Rigidized | Asp. | tional |
| | | state | state | Catheter −20 F. | 20 F. |
| without | Proximal | 2.09 | 55.6 | 1.35 | 4.39 |
| obturator | Distal | | | 0.92 | 1.67 |
| With | Proximal | 2.7 | N/A | 1.35 | 6.81 |
| obturator | Distal | | | 0.92 | 2.17 |

The EI stiffness is the stiffness (ratio of bending moment to curvature change) one would get for a homogenous material shaft (like a wire) with a Young's modulus of E and a cross-section corresponding to I. The rigidizing aspiration sheath catheter tested are heterogenous in structure, but it is still valid to characterize them with an equivalent EI parameter.

Recall the rigidizing aspiration sheath catheter may be first delivered up the vessel in its flexible state over its own obturator. Its composite EI stiffness (2.7 lbf-in²) is well below that of the proximal portion of the commercial catheter when it is delivered with its obturator (EI stiffness is 6.81 lbf-in²) and slightly higher that the stiffness of the distal portion of the commercial catheter (EI stiffness is 2.17 lbf-in²). Note the distal portion of the rigidizing aspiration sheath catheter is not necessarily intended to reach the target anatomy, so that slight increase of stiffness (2.7 lbf-in² vs 2.17 lbf-in²) isn't really a disadvantage. Once the rigidizing aspiration sheath catheter reaches its target location and accommodates to the anatomical curvature, it is rigidized and the obturator is removed after which the EI stiffness becomes 55.6 lbf-in², which is significantly higher than the stiffness of the commercial catheter with its obturator removed. Even though it is 12.7 times stiffer than the commercial catheter (proximal region), it is applying only minimal or negligible compressive tractions to its surroundings in contrast to the commercial catheter which has stored elastic energy needed to keep it in a curved shape and is compressively impinging on its surroundings.

An aspiration catheter may then be delivered up the rigidized rigidizing aspiration sheath catheter (on its own obturator, which may be removed after reaching target). The distal portion of the aspiration catheter may protrude beyond the distal end of the rigidizing aspiration sheath catheter and traverse the tortuous pulmonary artery anatomy.

The EI stiffness for the distal portion of the Aspiration catheter (0.92 lbf-in²) is significantly lower than the EI stiffness for the distal portion of the commercial catheter (1.67 lbf-in²). This means the aspiration catheter will have an advantage over commercial catheter when accessing tortuous anatomy.

In general, the catheters described herein (e.g., the rigidizing aspiration sheath catheter) may be any appropriate length, including up to more than 100 cm (e.g., 110 cm or longer, 115 cm or longer, 120 cm or longer, 125 cm or longer, 130 cm or longer, 135 cm or longer, etc.).

### Robotic Apparatuses

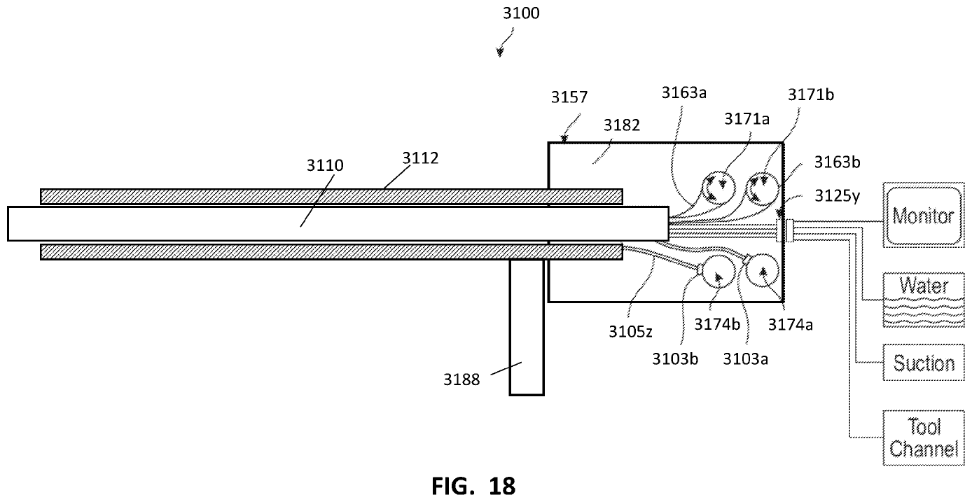

As mentioned above, the apparatuses described herein may be configured as part of a robotic system or for use with robotic apparatuses. In some examples the rigidizing aspiration sheath catheter system may include an outer tubular member that is robotically controlled, such as a robotically controlled overtube and/or endoscope assembly. FIG. 18 (not to scale) shows a schematic examples of an apparatus 3100 configured as a rigidizing aspiration sheath catheter system that may be robotically controlled. The rigidizing aspiration sheath catheter system may include an elongate flexible body having a plurality of layers, including a rigidizing layer and a bladder layer that is configured to transition the rigidizing layer between a flexible state and a rigid state, and a lumen extending through the elongate flexible body as described herein. The rigidizing aspiration sheath catheter may include a hemostasis valve region 3188 extending proximally from the elongate flexible body, the hemostasis valve region may include a housing having a central bore that is continuous with the lumen, and comprising an annular seal within the central bore and at least one actuator movable coupled to the housing and configured to open and close the annular seal or to seal around a device positioned within the central bore, wherein the at least one actuator is sized and shaped to be used by one hand of a user.

In the system 3100 in FIG. 18, the rigidizing aspiration sheath catheter may be configured as the outer 3112 or inner endoscope 3110. The overtube and inner endoscope can be separately or collectively be robotically controlled or manipulated (e.g., steering, movement, rotation, etc. including in some examples, rigidizing). As shown in FIG. 31, the outer overtube 3100 and the inner endoscope 3110 may be terminated together into a common structure, such as a cassette 3157. The outer overtube 3100 can be movable with respect to the endoscope 3110 by rotation of a driver mounted to the cassette 3157. The system may include actuators 3171a, 3171b that may connect to cables 3163a,b respectively, to steer (e.g., bend or deflect) the tip of the endoscope 3110 (and/or outer overtube 3112). Other steering mechanisms (e.g., pneumatics, hydraulics, shape memory alloys, EAP (electro-active polymers), or motors) are also possible. The cassette 3157 can further include bellows 3103a, 3103b that may connect to the pressure gap of the endoscope 3110 and the overtube 3112, respectively to drive fluid through pressure lines 3105z, in variations for either the endoscope and/or the overtube that are configured to rigidize when pressure is applied. As shown in this example, the cassette 3157 can include eccentric cams 3174a,b to control bellows 3103a,b. Alternatively, one or more linear actuators can be configured to actuate the bellows. As another alternative, the devices can be rigidized and de-rigidized through one or more pumps or pressure sources (e.g., via pressure line 3105z).

### Preventing Hemolysis

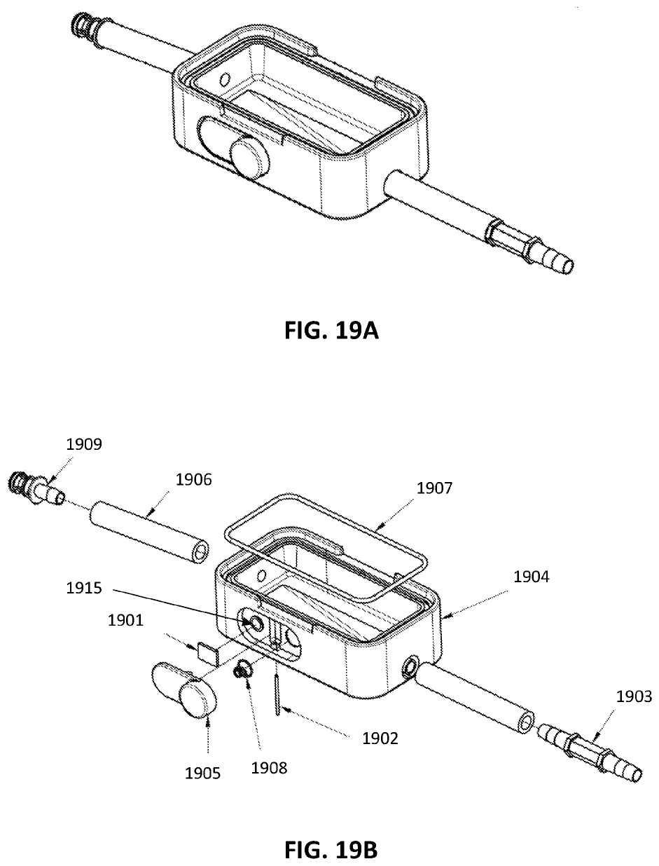

In general, these methods and apparatuses may also be configured to prevent or eliminate hemolysis during the procedure. For example, any of these apparatuses and methods may infuse air into the clot capture container at a control rate (e.g., more slowly/gradually than simply releasing/opening the system). This can be done by modulating diameter of hole beneath the venting button. FIGS. 19A-19B illustrate another example of a clot capture chamber 1900 configured to minimize hemolysis. In FIG. 19, the clot capture chamber includes a common rotator seal 1901, a dowel pin 1902, a barb fitting 1903, a clot capture body 1904, a release button 1905, tubing 1906, o-ring 1907, a conical spring 1908, a barb fitting (male) 1909, and sealing connectors 1910, 1911. By reducing diameter of the opening 1915 controlled by the button 1905, e.g., to a range of 0.020″ to 0.100″ slowed airflow in a way that reduces hemolysis.

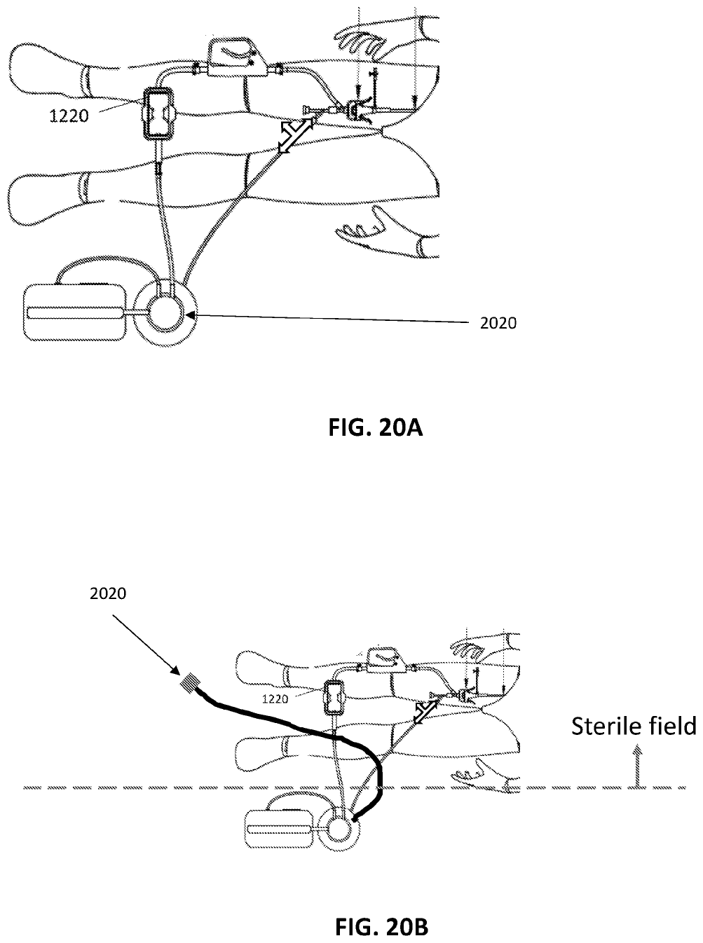

In any of these apparatuses a venting button may be positioned on top of the reservoir (e.g. the blood capture/filter chamber 1222). The venting button could be similar to the one shown in FIG. 19B in the clot capture chamber, with an associated hole/orifice whose diameter is in the range of, e.g., 0.020″ to 0.100″. When it is time to eliminate vacuum from the interior this button 2020 may be pressed so that air leaks into top of blood capture/filter chamber 1222. This is illustrated in FIG. 20A. As a consequence, there would be no rushing of air and blood in the tubing. Some blood may stay behind in the clot capture device, but overall there should be a reduction in hemolysis. In some examples a tube may extend from the reservoir to the sterile field, and the venting button 2020 may be pressed on the sterile field, as illustrated in FIG. 20B.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-32. (canceled)

33. A method for removing clot material, the method comprising:
advancing a rigidizing aspiration sheath catheter in a vessel while the rigidizing aspiration sheath catheter is in a flexible state so that a distal end of the rigidizing aspiration sheath catheter is near a clot material within a vessel, wherein the proximal end of the rigidizing aspiration sheath catheter comprises a hemostasis valve portion;
transitioning the rigidizing aspiration sheath catheter from the flexible state to a more rigid state; and
aspirating through the rigidizing aspiration sheath catheter in the more rigid state to remove the clot material.

34. The method of claim 33, further comprising:
inserting an aspiration catheter through the rigidizing aspiration sheath catheter,
extending the aspiration catheter distally out of the rigidizing aspiration sheath catheter proximate to the clot material with the rigidizing aspiration sheath catheter in the more rigid state; and
aspirating through the aspiration catheter.

35. The method of claim 33, wherein transitioning the rigidizing aspiration sheath catheter from the flexible state to a more rigid state comprises applying pressure to one or more layers of the rigidizing aspiration sheath catheter.

36. The method of claim 33, further comprising applying aspiration through the rigidizing aspiration sheath while aspirating through the aspiration catheter.

37. The method of claim 33, further comprising observing a clot material aspirated within a window of a clot capture chamber connected in-line with a rigidizing aspiration sheath catheter.

38. The method of claim 33, further comprising coupling a vacuum line to the proximal end of the rigidizing aspiration sheath catheter.

39. The method of claim 33, wherein aspirating through the rigidizing aspiration sheath catheter comprises activating a hand-triggered activation valve that is in-line with a vacuum line coupled to the rigidizing aspiration sheath catheter.

40. The method of claim 34, wherein extending the aspiration catheter distally out of the rigidizing aspiration sheath catheter comprises advancing the aspiration catheter distally without the use of a guidewire.

41. The method of claim 33, wherein extending the aspiration catheter distally out of the rigidizing aspiration sheath catheter comprises advancing the aspiration catheter comprises steering a distal end of the aspiration catheter while the rigidizing aspiration sheath catheter in the more rigid state.

42. The method of claim 33, further comprising repositioning the rigidizing aspiration sheath catheter within the vessel by converting the rigidizing aspiration sheath catheter into the flexible state, moving the rigidizing aspiration sheath catheter within the vessel, and rigidizing the rigidizing aspiration sheath catheter.

43. The method of claim 42, wherein moving the rigidizing aspiration sheath catheter comprises moving the aspiration sheath catheter within the vessel with an obturator within a lumen of the rigidizing aspiration sheath catheter.

44. The method of claim 33, wherein advancing the rigidizing aspiration sheath catheter to the treatment location comprises advancing a distal end of the rigidizing catheter to a pulmonary artery of a patient.

45. A method for removing clot material, the method comprising:
advancing a guidewire through a vessel;
advancing a rigidizing aspiration sheath catheter over the guidewire so that a distal end of the rigidizing aspiration sheath catheter is near a treatment location in the vessel;
transitioning the rigidizing aspiration sheath catheter from a flexible state to a more rigid state by applying or removing pressure within a wall of the rigidizing aspiration sheath catheter; and
applying aspiration through the rigidizing aspiration sheath catheter to remove clot material from the vessel wherein the rigidizing sheath catheter is held in a fixed location within the vessel while applying aspiration.

46. A method for removing clot material, the method comprising:
advancing a guidewire through a vessel;
advancing a rigidizing aspiration sheath catheter over the guidewire so that a distal end of the rigidizing aspiration sheath catheter is near a treatment location in the vessel;
transitioning the rigidizing aspiration sheath catheter from a flexible state to a more rigid state by applying or removing pressure within a wall of the rigidizing aspiration sheath catheter;
extending an aspiration catheter through the rigidizing aspiration sheath catheter and distally out of the rigidizing aspiration sheath catheter with the rigidizing aspiration sheath catheter in the more rigid state; and
aspirating through the aspiration catheter to remove a clot material from the vessel.
